(19) United States
(12) Patent Application Publication
(10) Pub. No.: US 2014/0108983 A1
(43) Pub. Date: Apr. 17, 2014

(54) GRAPHICAL SYSTEM FOR COLLECTING, PRESENTING AND USING MEDICAL DATA

(71) Applicant: Karen Ferguson, (US)

(72) Inventors: Dobkin William R., Newport Beach, CA (US); Hugh Ferguson, Rancho Santa Margarita, CA (US); Christopher Wiggins, Aliso Viejo, CA (US)

(21) Appl. No.: 13/564,938

(22) Filed: Aug. 2, 2012

Related U.S. Application Data
(60) Provisional application No. 61/589,365, filed on Jan. 22, 2012.

(51) Int. Cl. G06F 19/00; G06F 3/0484; G06F 3/0482; A61B 5/00

(52) U.S. Cl. CPC G06F 19/3406; A61B 5/7435; G06F 3/0484; G06F 3/0482
USPC 715/771

(57) ABSTRACT

Briefly, a medical data system collects data concurrent with performance of a medical procedure. In one example, the medical procedure is a surgery, and a surgical support system collects and presents surgical information concurrent with the surgery's progress. A graphical display in the operating room is used to capture in near real time, what happens in the operating room. Medical personnel in the operating environment use the surgical system to capture detailed information regarding the surgery. The surgical system allows the medical personnel to graphically record key data, which enables a surgical report to be promptly generated. In this way, the surgical system enables the near real time collection and presentation of surgical data, and ensures the data and the surgical report is accurate and complete.

### RELATED APPLICATIONS

This application claims priority to U.S. patent application Ser. No. 12/455,825, filed Jun. 8, 2009, and entitled “Medical and Surgical Process System and Method,” and to U.S. provisional patent application No. 61/589,365, filed Jan. 22, 2012, and entitled “System and Method for Collecting, Presenting, and Using Surgical Information,” both of which are incorporated herein as if set forth in their entirety.

### FIELD OF THE INVENTION

The invention relates generally to computer systems and processes for collecting, presenting, and using medical information. More particularly, in one example, the invention relates to a system and method for collecting surgical data during a surgical operation, presenting the surgical data to the surgical team, and enabling immediate use of the surgical data to support patient well being and the related medical businesses.

### BACKGROUND

With the increasing cost and rising concerns related to patient care and the healthcare system's economic stability, one of the most important areas of concerns is the use of information and patient data along with effective and efficient methods of comparative effectiveness evaluations. In the United States, for example Hospital IT (Information technology) budgets are now estimated to be in the 12%-14% of hospital cost, up substantially from recent prior years. Unfortunately, due to the lack of wide-spread adoption of business technologies in the healthcare industry; today's health industry is currently reliant on excessively manual and outdated methods. These methods not only are inefficient and expensive, but they are prone to mistake, error, and cannot support effective decision management.

Healthcare is one of the hottest topics in the world today when it comes to a healthy population and economy. For surgical practices in particular, the collection, tracking and storing of pertinent medical data is a subject that must be dealt with immediately in order to manage costs and utilize data. Further, current process and procedures are subject to significant time delays, omissions, and errors. For example, a surgeon may conduct an operation, and at a later time try to remember operative details while dictating his or her operative report. Even later, the dictation has to be transcribed, introducing further delay and possibility of error.

Amazing advances have been made in the past decade in medical care, instrumentation, and technology, yet the past cumbersome business models and practices continue to threaten the future of the medical industry. For decades, an antiquated process of collection and management of medical and surgical data has been used, thereby creating a multitude of problems as described more fully below.

Paper records cannot be searched easily. Electronic queries are extremely costly and time consuming, making them practically impossible to use to support patient care or business decisions. For example, it is difficult to locate in which particular patient a specific implant has been used, and in many cases the information simply does not exist in any form. In others, the data is so scattered and disjointed that the implant or patient cannot be economically tracked. Accordingly, even if surgical results and patient information have been recorded, due to the limitations of traditional EMR (electronic medical records), it is not feasible to track implants. In this way, if a vendor or government agency recalls a particular implant for safety reasons, it is most often impossible to contact or even identify the particular patients that have those faulty implants.

Presently, data searches require significant manpower to sift through paper records to find surgical information, medical records and implant history. At best, such searches are slow, time consuming, usually lack complete information, and are prone to substantial error and machine or human error. Medical and surgical records are only as complete and accurate as entered by a technician. There is no guidance or standardization to ensure the proper data is entered, nor are the entries always done concurrent with a surgery or use of an implant or biomaterial. Such after-the-fact entry often times leads to incorrect and incomplete records of the medical procedure, including what implants and biomaterials were put in the patient's body.

Further, there is no reliable or readily accessible current tracking or database of implants or biomaterials. This makes recalled items nearly impossible to locate and surgical outcomes immeasurable. For manual or semi-automated process that do exist, they often run afoul of medical privacy regulations, such as HIPPA in the United States.

Revenue is lost as preexisting conditions or complications are often overlooked in surgical reporting. Inconsistent billing practices make collections difficult to manage. Tracking and replenishing of surgical instruments and implants also relies on a paper system leaving the opportunity to lose or misplace expensive devices. As a consequence, the current system is unaccountable and prone to fraud and abuse. Current data collection practices also neglect full compliance with governmental guidelines for implant and biomaterial tracking, and are not prepared for future initiatives requiring more detailed accounting of parts, infections, and medial outcomes.

In the past, these issues were less significant as the medical business was structured to absorb or pass-on increased costs and spending. However, revenues are down in the industry today, while the cost of care continues to rise, driving the need for new cost effective methods. With all of the changes in the economy, including substantial job loss and organizational restructuring, the medical industry cannot continue to provide adequate medical care without a major change in the infrastructure and way data is collected, processed, and used.

Currently the work flow and logistics of this information is a manual process with the reduplication of paperwork and limited, if any, use of electronic technologies on the front end. Sales representatives are completing usage forms and pricing by hand and providing this information to hospital administration several days following the time of medical and or surgical treatment. Sales representative then call or fax usage information regarding vendor billing and the creation of an invoice. In order for the vendor to obtain a PO they rely and wait for the sales representative to call or email the hospital for this information followed with the sales representative calling in or emailing the PO to the vendor and the vendor subsequently sending out the invoice to the treatment facility. The manual system is slow, inefficient, and susceptible to errors, incompleteness, abuse, or outright fraud.

A shift in focus has recently occurred in the medical industry, making efficiency, logistics, cost of care, and infrastructure remodeling significant current priorities. The critical problem lies with the “effective use” of medical data and “comparative effectiveness” of patients care and financial cost. There is currently no effective ‘front-end’ method to accurately and timely capture critical data, and in particular surgical data. Old carbon-copy paperwork is being replaced with newer systems for electronic storage, but these new EMR systems are already proving to cultivate the traditional garbage-in, garbage-out structure with enormous up-front costs of development and implementation, while failing to addressing the underlying fundamental failures of the current systems. Without changing the ‘front end’ collection process, the industry is unable to provide the needed resource management applications, useful information, or the necessary analytics to comply with impending federal regulations. The ‘front end’ data collection process is the key to making the medical information available, complete, useful and effective in today's cost of care and performance models. Therefore there exists a need for a system and method that can provide complete and systematic data collection at the time a medical procedure, such as a surgery, is done to enable timely, accurate and usable medical information.

### SUMMARY

The invention of the present disclosure is directed to a system that collects data concurrent with the performance of a medical procedure on a patient. In one example, the medical procedure is a surgery, and a surgical support system is used to collect and present surgical information concurrent with the surgery's progress. The surgical support system has a graphical display in the operating room that is used by a technician, nurse, or doctor to capture, in near real time, what happens in the operating room. Generally, the surgical support system allows medical personnel to select a medical process that is to be performed, such as spine surgery, and then display an anatomic rendering for the specific area of the patient being operated on. Medical personnel in the operating environment use the surgical system to capture detailed information regarding the surgery, such as what tools, parts, and implants are used, the placement of implants, and surgical removal and reconstruction procedures. The surgical system enables the medical personnel to graphically record and confirm key data, which then can be viewed and approved by the surgical team. In this way, the surgical system enables the near real time collection and presentation of surgical data, and ensures the data is accurate and complete.

The surgical system is also constructed to avoid fraud and over-billing. For example, the surgical system has an intelligent anatomic rendering that restricts the placement and number of implants according to pre-defined rules. In this way, the system will not allow an excessive number of implants or parts to be used, and only parts that have been properly placed can be used to generate billings or invoices. Further, the concurrent display of parts and processes used provides a level of transparency and accountability that has never before existed in the field of medical data control. Indeed, the surgical system can even be configure to send warnings and alerts to various hospital, insurance, or regulatory personnel if someone attempts to generate an invoice for an improperly placed or an unauthorized part or procedure.

Since medical data, such as surgical data, is timely, completely, and accurately captured, the surgical system also enables a surgeon to quickly and efficiently generate an operative report, without waiting for transcription or form documents. Indeed, since the doctor or surgeon can confirm, even while performing the medical procedure, that the parts, medical process, personnel, complications, and outcomes are accurately recorded, the surgeon can immediacy proceed to issue the surgical report or other completion report. Since the surgical system is also populated with billing codes and patient information, it can automatically assist with ICD10 hospital coding and billing, and generate the data to bill private insurance or government agencies in a timely manner. Implants and biomaterials are granularly tracked, so the data may be used to populate a complete and accurate implant registry, thereby enabling follow-up and identification in the case of recall. This can include data support for a “National Implant Registries” for all implanted products (inclusive of medical devices, biologics, or tissue), something which is mandated by the U.S government.

### BRIEF DESCRIPTION OF THE DRAWINGS

FIG. 1 is a flowchart and data diagram for a medical data system in accordance with the present invention.

FIG. 2 is a diagram of a medical data system in accordance with the present invention.

FIGS. 3A and 3B are screen illustrations of a selection process for a medical data system in accordance with the present invention.

FIGS. 4A and 4B show an input screen and flowchart for adding a patient to the medical data system in accordance with the present invention.

FIG. 5 shows a surgical operative report generation system for a medical data system in accordance with the present invention.

FIG. 6 shows input screens for a medical data system in accordance with the present invention.

FIG. 7 shows input screens for pre and post-operative diagnostics for a medical data system in accordance with the present invention.

FIGS. 8A and 8B show input screens for disposables and biologicals for a medical data system in accordance with the present invention.

FIGS. 9A and 9B show input screens for sterilization sources and global procedures for a medical data system in accordance with the present invention.

FIG. 10 shows a graphical and tabular selection process for initiating a spinal surgery for a medical data system in accordance with the present invention.

FIG. 11 shows an anatomical representation of a spine and data flow for a graphical spinal selection process for a medical data system in accordance with the present invention.

FIG. 12 shows a flowchart of a process for selecting spinal levels for a medical data system in accordance with the present invention.

FIG. 13 shows graphical and tabular selections of removal procedures for spinal surgery done on a medical data system in accordance with the present invention.

FIG. 14 shows spine remove images for a medical data system in accordance with the present invention.

FIG. 15 is a flowchart of beginning a removal process for a medical data system in accordance with the present invention.

FIG. 16 shows a series of flowcharts for selecting specific removal procedures for a medical data system in accordance with the present invention.

FIG. 17 shows a flowchart for a removal procedure for a medical data system in accordance with the present invention.

FIG. 18 shows a graphical layering system for performing reconstruction procedures for a medical data system in accordance with the present invention.

FIG. 19 shows a graphical layering system and associated rules for use with a graphical overlay system for a medical data system in accordance with the present invention.

FIG. 20 shows a graphical overlay system and hardware selection input screen for a reconstruction process for a medical data system in accordance with the present invention.

FIG. 21 shows a flowchart for performing reconstruction using a graphic overlay system for a medical data system in accordance with the present invention.

FIG. 22 shows a graphical overlay system for collecting graphical data for a medical data system in accordance with the present invention.

FIG. 23 shows an anatomical representation having overlaid hardware for a reconstruction process for a medical data system in accordance with the present invention.

FIG. 24 shows an input screen for a reconstruction process for a medical data system in accordance with the present invention.

FIG. 25 shows a reconstruction process for a medical data system in accordance with the present invention.

FIG. 26A shows a flowchart of a graphical data collection system for a medical data system in accordance with the present invention.

FIG. 26B shows a flowchart for a reconstruction process for a medical data system in accordance with the present invention.

FIG. 27 shows input screens for fluids and surgeon notes for a medical data system for a medical data system in accordance with the present invention.

FIG. 28 shows an input screen for anesthesia and medications for a medical data system in accordance with the present invention.

FIG. 29 shows an input screen for identifying complications in a medical data system in accordance with the present invention.

FIG. 30 shows additional input screens for inputting data into a medical data system in accordance with the present invention.

FIG. 31 shows additional hospital information screens for a medical data system in accordance with a medical data system in accordance with the present invention.

FIG. 32 shows an input screen for obtaining patient or legal consents in a medical data system in accordance with a medical data system in accordance with the present invention.

FIGS. 33A-D show an example of an automatically generated surgical operative report for a medical data system in accordance with the present invention.

FIG. 34 shows another example of a type of surgery that may be done with the medical data system in accordance with the present invention.

### DESCRIPTION OF PREFERRED EMBODIMENTS

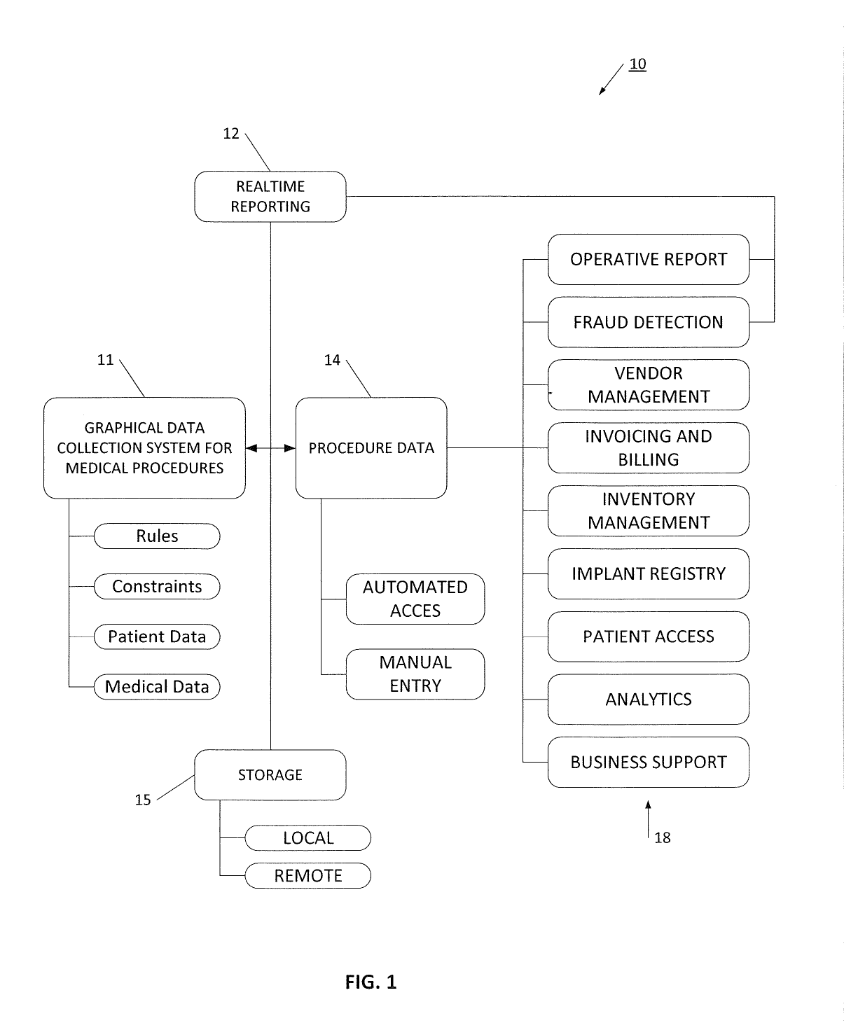

Referring now to FIG. 1, an example of a medical data system 10 is illustrated. A medical data system 10 includes a graphical data collection system 11 for accurately collecting medical procedure data in real time or near real time concurrently with the performance of the medical procedure. The data collected from the real time graphical data collection system 11 is then used to provide information and data to support various aspects of the medical data system 10. For example, the graphical data collection system 11 may collect data in the medical environment where the medical procedure is being performed, and provide a real time or near real time reporting 12 of that information. It will be appreciated that the graphical data collection system 11 may include a touch screen, keyboard, mouse, tablet computer, display, or other graphical data input device. The real time reporting 12 may be accomplished using a display, graphical display, lights, alarms, or other device to present medical data into the environment where the medical procedure is being performed. In one example, the graphical data collection system 11 is used by a surgical nurse in an operating room for real time collection of surgical data. Information reflected in the surgical procedure is then displayed in real time 12 to members of the surgical team. It will be appreciated that other real time or near real time reporting may be made to others outside the area where the medical procedure is being performed, for example.

The medical data system 10 also has procedure data 14 to support the collection and presentation of medical procedure data. For example, procedure data 14 may include specific information regarding the medical procedure to be performed, or may include data specific to the patient, medical procedure environment, medications, or personnel involved in the procedure. It will be understood that the procedure data may in some cases be automatically accessed from other existing electronic databases, may be manually input prior to the medical procedure, or may be manually entered in real time or near real time during the medical procedure. The data is stored in storage devices 15, which may be local to where the medical procedure is being performed, may be in a local area network environment, or may be stored remotely using some wide area access systems.

Once the real time data and procedure data has been collected regarding the medical procedure, the data may be used to drive certain reports and business aspects 18 of the medical data system. For example, the data may be used to automatically generate operative reports, detect fraud in real time, manage vendors, provide for highly efficient invoicing and billing, provide for inventory management for the hospital or medical provider, may support a national implant registry, may provide for patient access to medical data, may provide analytics to evaluate the effectiveness and medical procedures, or may provide medical business support for the medical practitioner or hospital. It will be understood that other uses of the data may be found. Importantly, it is the timely, accurate, and complete collection of data by using the graphical data collection system 11 that enables the efficient and effective management of the medical business, as well as facilitating improved delivery of healthcare to the patient.

Several general types of information become readily available and usable because of the accurate and timely data collected by the medical data system 10. For example, the system 10 can accurately collect and present the medical procedure performed and what the clinician or medical provider actually did; what products were use in the procedure, including any implants or medical devices used; the cost of the products used; the outcome of care and how well the patient did during and after treatment; cost metrics of the provider, the hospital, or the specific procedure; and the overall effectiveness of medical procedures. It will be appreciated that other applications may be used consistent with this disclosure.

The timely and accurate process of collecting data on the front end is the foundation for the medical data system 10 and related business models it supports. The system 10 allows for nearly unlimited analytics and data management. The medical data system 10 revolutionizes the process of data collection and allows for the “real time” or near real time collection of medical procedure information and stores this into a specifically designed storage infrastructure, which allow for flexible analytics, searchability of data within a hospital or related to a single patient, and also throughout the entire network of treatment facilities, including any number of patient or analytic parameters.

The system 10 allows for distributed or cloud-base collection of any surgical information, surgical removal procedural, reconstructive procedures, implant or medical device location, medical device to patient linking, implant or medical device combined-use tracking, medical device or implant identification (part numbers) with or without lot numbers, tracking or sterilization sources, linking part identification, lot number (ID), sterilization sources, medical device and implant sources.

The medical data system presents structured information and instructions through the graphical data collection to guide the operators through the data entry process and to assure that data is consistently and uniquely identified. In addition, the ability to collect one of many types of surgical procedures including but not limited to: spine, cardiac surgery, interventional cardiology, hip surgery, and knee surgery from various treatment locations or hospitals and being able to use local or wide area networks to link these into a single “Patient Medical Profile” is unique to the medical data system.

Graphic interactive displays may assist technicians to place and to record surgical implants contributing to a more complete and accurate surgical record. In addition, the process of the transfer graphic interface modifications or surgical representation into a text form (i.e. operative, clinical, or business reports) and describe the procedure or implant or medical device and or location in a text form is unique to the medical data system.

The collected medical and surgical data may be stored in a local or wider area computing system and can be accessed anywhere at any time. Accessible relational tables, if used, can afford strong search capabilities. Today, records and EMR are stored as “flat/pdf” type images which are scanned and stored after the surgery has been completed. These images are static and limited in their abilities for searchable, report creation, analytics and any customized data specific queries because they are not digitally indexed. That is, there is no practical way to provide relationships between documents and files. Whereas in the disclosed medical data system, methods and process enable the unlimited analytic and search ability of any point of data, as the data may be stored electronically into a web-based database system at the time of surgery as an individual piece of data. This process and method is advantageous as the web allows for the linking of all individuals patient data into global patient records. It will be understood that other storage and access architectures may be used consistent with this disclosure.

The specific information collected by the medical data system may be stored in both temporary and permanent data tables. The data storage process may be different during the time of data collection in comparison to the time when the data collection for the medical treatment is completed. It will be understood that data may be stored and accessed from any manner of data sources, such as data bases, text files, data structures, libraries, relational files, or flat files. For ease of explanation, the data source typically is described herein in the form of a database, but it will be understood other data-management techniques and processes can be used. The real time data may be stored in temporary relational data table and thereby allows a user to correct, add or delete the data prior to committing to more permanent storage. These data tables can be manipulated until the completion of the data collection process, when the operator will activate a commit, store, or save function to indicate that the information in temporary storage is complete and correct. This simple selection process converts the temporary relational data tables into a permanent relational data table, which them becomes part of a permanent, unalterable record that is digitally optimized for integration and use.

Once front-end data is entered, detailed reports may be automatically generated. Unlimited report creation is a powerful aspect of the medical data system. In one example, the medical data system allows tracking of costs versus outcomes, making administrative decisions less obfuscated, more meaningful, and more straight forward. The ability to track information and the meaningful presentation of the data helps organizations comply with federal and state initiatives, as well as improve patient care while reducing costs.

The medical data system can enable the implantation of an efficient implant registry and can finally make surgical implants searchable based on their lot and part numbers, and provide detailed surgical information, including their location in a patient's body. Currently in the US there is no national implant registry. The medical data system has the capability to track and link the following via an electronic and or web/cloud based system: patient identification, implants, procedure performed, lot numbers, any other implant identification, the location of the implant in the body, the source of the implant and the sterilization history of the implant. It will be appreciated that other information may be stored and accessed as needed. Any recalls sent from the registry may be sent to the hospitals that performed the actual surgery. The hospital and patient will have the only access to the patient's personal data, and can contact the patient consistent with established privacy constraints.

The implant registry can be used by government, hospitals, patients, vendors, manufacturers, and tissue banks to enable the effective identification of newly recalled or revised implants, and remove them from inventories prior to being used. Additionally, this type of registry would allow vendors, manufacturers, and tissue banks to inform treatment facilities of recalls and other product-related issues or information and provide a set of identifiers to the hospital or doctor that would enable them to contact the specific patient with the faulty implant.

Vendors will now have the ability to track medical devices, eliminate fraud, and improve business processes. Currently it is routine for sales representative to be in surgery for 4-8 hours and track the implants with triplicate carbon copy paperwork and to call in for replenishment to the supplier using the phone and calculator to add up the total of the hospital bill. Today implant providers are unable to effectively tracking implants and link part numbers, lot numbers, or patients. Under present systems, a surgery might cost $300,000, with $50,000 or more in hardware, medical devices and implants placed in the patient's body. Presently there is no way to track any of the hardware, or even assure the patient has received all the implants or hardware they purchased. The medical data system includes the inter-operative capability of collecting implant information—biological and hardware, medical devices of any kind—in an electronic and real time capture and database storage with the ability to accurately and effectively track, link, and identify all of implants and medical devices put in any patient.

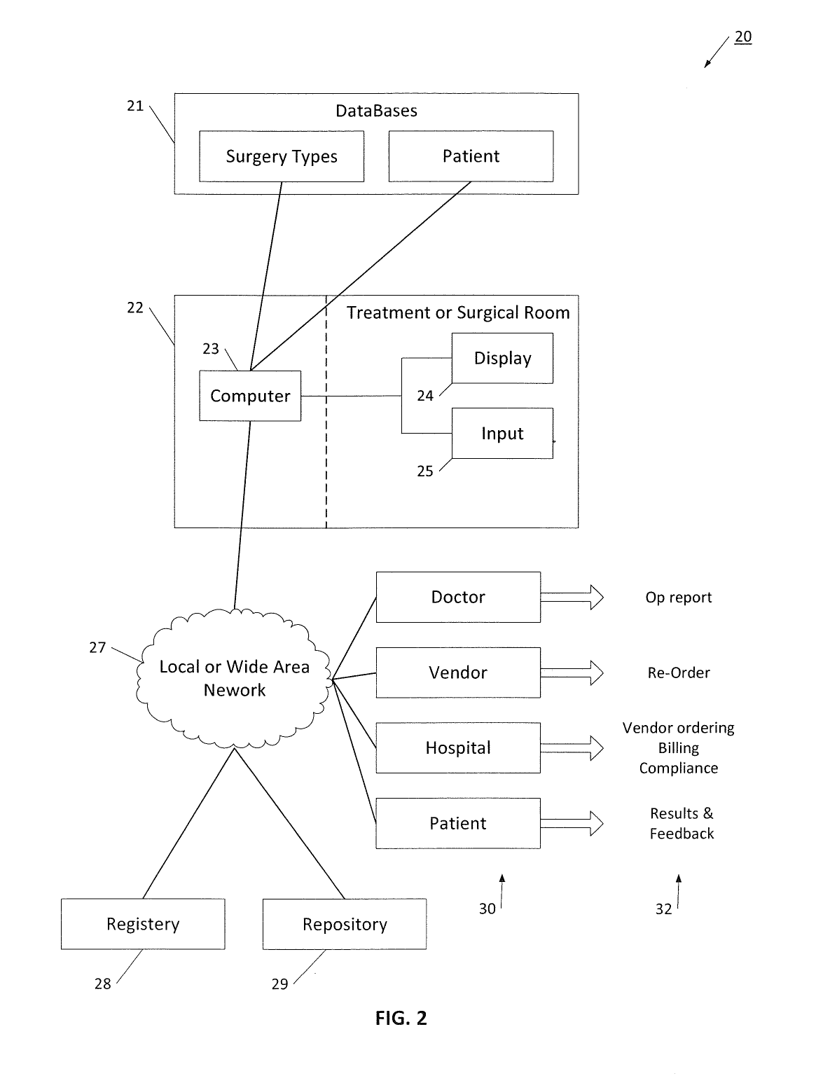

Referring now to FIG. 2, an example of a medical data system 20 is illustrated. The medical data system 20 may be, for example, used with regard to providing a medical treatment or procedure to a patient. For simplicity, an example of providing a surgical produced will be used, although it will be appreciated that many other medical treatments and procedures are contemplated consistent with this disclosure.

The medical data system 20 is provided to enable timely and accurate collection and presentation of medical information in close association with the performance of the medical procedure. In this way, the medical procedure information is collected in near-real time, and can be verified by the medical personnel present at the time. As illustrated in FIG. 2, a computer 23 is provided that has a display 24 for use in the treatment or surgery room 22. Medical personnel, such as a nurse, technician, or doctor uses one or more input devices 25 to collect and record medical information as the procedure is performed.

For a surgery procedure, the computer operator selects a particular surgery to be performed from a set of surgery types available in the system database 21. Responsive to the selected surgery type, the display 24 shows a graphical representation of the surgery target, such as a spine portion or a heart. Information specific to the patient is also accessed, which may also be displayed to the surgical team. Although FIG. 2 shows a standard computer configuration, it will be understood that other devices, such as laptops, wireless appliances, smartphones, or PDA's may be used as part of the system.

As the team prepares for surgery, information specific to the surgery is collected. For example, information may be collected regarding the location of the operating room and personnel present for the surgery. The biomaterial, implants, and tools for the surgery may be identified and confirmed as being properly sterilized and handled. Once the surgery begins, the computer operator annotates the graphical representation to show tissue removal, reconstruction, and placement of implants or parts. The annotations are done graphically or with the use of guided input screens. In one example, the display builds a graphical representation of the surgery. The surgical team can view the display as the image is annotated, so they can provide immediate confirmation that surgical information is being accurately captured. Further, the surgical system maintains a list of parts, implants, and biomaterials that may be used for the selected surgery type, so the system assures that only authorized parts, implants, and biomaterials can be used.

The graphical annotation is also intelligent, that is, the surgical system only allows parts, implants, and biomaterials to be placed in defined quantities and at defined places. For example, for a spinal surgery type, the intelligent anatomical display permits placement of only authorized types of screws, and a certain quantity of screws at any predefined locations. If the operator attempts to position too many screws at a location, or place a screw in an unapproved location, then the system can be set to generate fraud warnings. Further, the immediate visibility on display 24 of any annotation to the surgical team also acts to reduce the occurrence of fraud.

The surgical team may also capture information regarding pharmaceuticals, complications, and outcomes during the surgery. In one use, the surgeon signs off on the surgery completion before leaving the operating room. In this way, the surgeon or doctor can complete the surgical report in an automated way using the complete and accurate information collected in the surgical room. The anatomical image, annotations, and data inputs all become part of the permanent record for that patient.

The medical data system 20 may use a wide-area network 27, such as the Internet, to enable interested and authorized parties 30 to access data, and to organize and view medical information reports 32. The wide area network may also interface an implant registry 28 for facilitating implant and tissue tracking.

Once the surgeon or doctor has approved that the procedure has been accurately capture, and has generated the surgical report, the hospital can use the information to re-order parts from vendors, pay vendors for the parts used, communicate invoices and billing information to insurance companies or government providers, or provide information to show regulatory compliance. Since the annotations, parts, implants, and procedures may be linked to specific standard billing codes, the generation of bills and invoices may be mostly automated.

Advantageously, the information regarding implants may be stored in a registry 28 in a manner that is compliant with privacy regulations, such as HIPPA in the US, but that still enables a hospital to know the specific implants it has consumed, and the specific patient that received the implant or biomaterial. In this way, if a vendor recalls an implant, the vendor can determine which hospitals have used the defective part, and the hospital can identify which specific patient has the implant.

As the use of the medical data system becomes more widespread, a substantial amount of useful medical data will be collected and stored in a repository 29. The data can provide key analytics and metrics for the medical industry, such as treatment efficacy, costs, and quality control. By way of example, a hospital can use the repository 29 to understand if its doctors are performing to industry standards or evaluate whether patients are satisfied with outcomes. The hospital may readily identify doctors that are underperforming, or that need training to perform procedures in a more cost effective way. In a more specific example, the data can be used to evaluate and compare the effectiveness of drugs or treatments.

At the time of surgery, healthcare providers may concurrently enter standard information into the system, rather than manually recording with paper and pen. This system, which may be a web 27 driven software system, is simple to use by allowing an operator to point and click to select parts, implants or biomaterials, and then position the selection onto an anatomical rendering of the patient and the specific surgical site. This information is intelligently captured and made useful as it transfers into our accessible relational database tables at nearly the same time as the medical procedure takes place. Completing this simple, yet critical step of electronically capturing real time information allows the medical data system to make the interpretations and specific data storage resulting in automated generation of the summary reports, business applications, analytics, and financial evaluations. Also included in the system is the ability to have the descriptions of data collected during surgery (selections) link to an alternative description which would show up on the operative report generation and or other reports. For example, during the data collection you may have this description for the “selection”: (i.e. “Removal of Lamina”). But on the Operative Report of other report generation this “selection” would have the ability to be described differently: (i.e. “L2 bilateral removal of lamina”).

The surgical system eliminates the extensive and costly manual charting by leveraging the power and accessibility of local and wide area networks to automate and bring forth advanced surgical data collection and functionality. Specific web-based applications may function as a clinical and business database creating a readily accessible portal accepting all patient pertinent information associated with a surgical procedure from the time of entering the hospital to the time of leaving the operating room. In particular, the surgical system substantially eliminates the need for waiting for dictation to be transcribed, instead allowing for accurate, complete, and near-real time capture of surgical information. In the special case where transcription is still desired, the surgical system allows the transcribed text to be stored and maintained.

As illustrated generally in FIG. 2, the medical data system 20 is capable of efficiently and accurately providing time sensitive information. For example, the system can generate electronic operative reports concurrent with a medical procedure, and may include graphical representations of the selected aspects of the procedure.

### Example of a Medical Data System

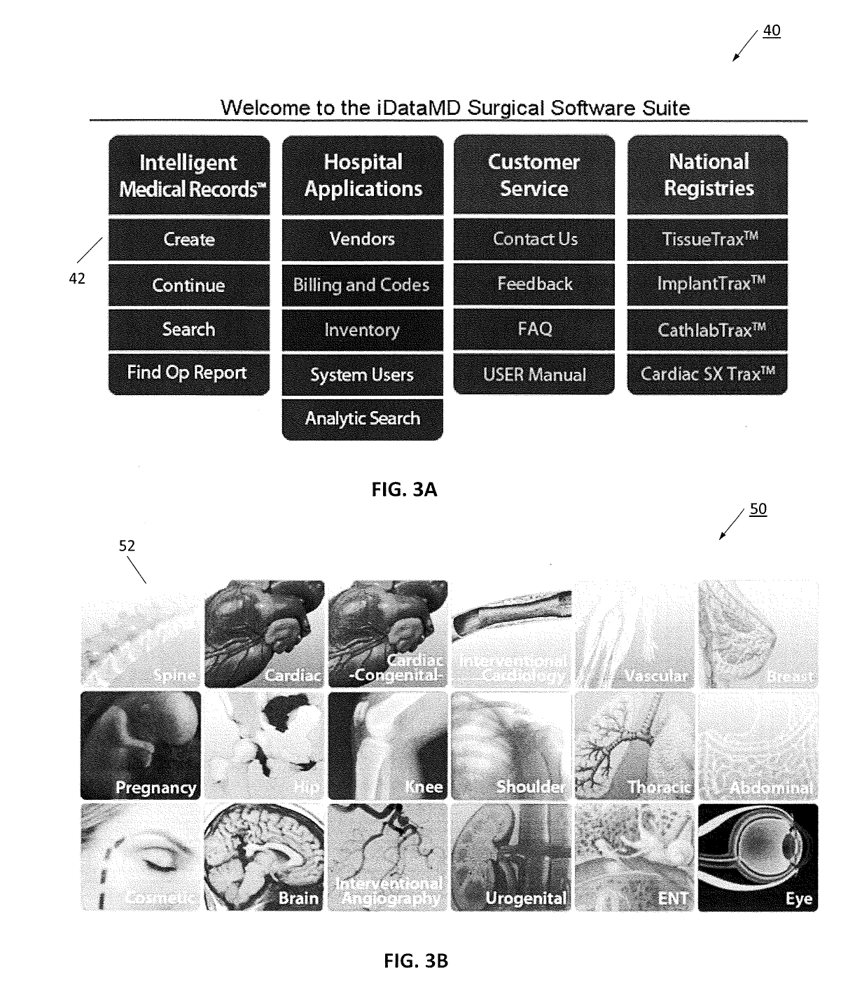

In FIGS. 3 through 33, an exemplary embodiment of a medical data system is illustrated. The medical system described in the following figures is similar to the general medical data systems 10 and 20 described with reference to FIGS. 1 and 2. In particular, the exemplary embodiment described in the following figures describes a medical procedure in the form of a surgery. It will be appreciated that other medical procedures may be used in accord with the claimed invention. The particular example illustrated uses a graphical computer input system positioned within the confines of a surgical room. Typically, the computer input is done by a surgical nurse or other medical personnel. A graphical display is also positioned within the surgical room for presenting the collected data to personnel in the surgical environment. In the illustrations of the following figures, the specific surgery is in the form of a spinal surgery. However, it will be appreciated that other types of surgery and medical procedures could be substituted consistent with this disclosure. Further, the following disclosure uses a local computer in or near the surgical room, which is connected to a local area network. The local area network has local storage, with the local system connected through a wide area network for sharing of information between hospitals and third parties. In one example, the wide area network is the Internet. Storage and communication architectures are implemented using known security processes and procedures.

Referring now to FIG. 3A an initial selection screen 40 for a medical data system is illustrated. As illustrated, initial screen 40 allows an administrator or other medical personnel to begin a medical procedure such as a surgery. Entry screen 40 has selection boxes 42 divided into four categories. It will be appreciated that the specific arrangement and selection of the functions may be set according to application specific requirements. In particular, input screen 40 allows for management of the medical procedure under the heading of “intelligent medical records.” It enables hospital administrators to manage vendors, inventory, and perform analytics under the heading of “hospital applications.” Input screen 40 further allows the end user to communicate with the data system manager through the “customer service” tab. Finally, the input screen 40 may contain links to third party registries for managing tissues, biometrics, and implants beneath the “national registry” heading. It will be understood that not all of these functions need to be implemented to have a medical data system operating in accord with this disclosure.

To initiate a new surgery, a medical personnel would select the “create” tab from entry screen 40. Upon selecting “create,” the user would be presented with the input screen 50 shown in FIG. 3B. Input screen 50 has several input buttons 52, each indicating a particular type of surgery. Screen 50 illustrates several types of surgeries including spine, cardiac, vascular, breast, pregnancy, hip, knee, shoulder, lung, eye, brain, as well as others. It will be appreciated that other types of surgery procedures may be included, as well as non-surgical procedures.

The surgical data system can accommodate many surgery types. Here, for example, the illustrated surgical data system allows surgery types such as spine, cardiac, breast, hip, and knee. It will be appreciated that nearly any type of surgery may be contemplated within the scope of the surgical data system. When selected, each of the surgery types links to a database containing predefined anatomical representations supportive of the selected surgery. For example, if spine is selected as the surgery type, a set of anatomical representations of the spine may be presented to a medical operator for further selection. In a similar manner, if cardiac is selected as the surgery type, then and anatomical representation of a heart or a portion of a heart may be presented.

A database, which may be stored locally or on a local or wide area network, also holds information regarding the specific surgery patient area. It will be appreciated that the database may be the same or separate from the database storing information related to the surgery types. Typically, the patient will be identified by an identification number, which associates the patient with known medical history, as well as information for the proposed surgery. Once a patient has been selected and a surgery type indicated, the anatomical representations become part of that patient's medical record. As will be described later, annotations and image representations are made on the anatomical representation corresponding to the surgical and medical procedures performed. Advantageously, this near real time and graphical collection of surgical data accurately and immediately becomes part of the patient medical record.

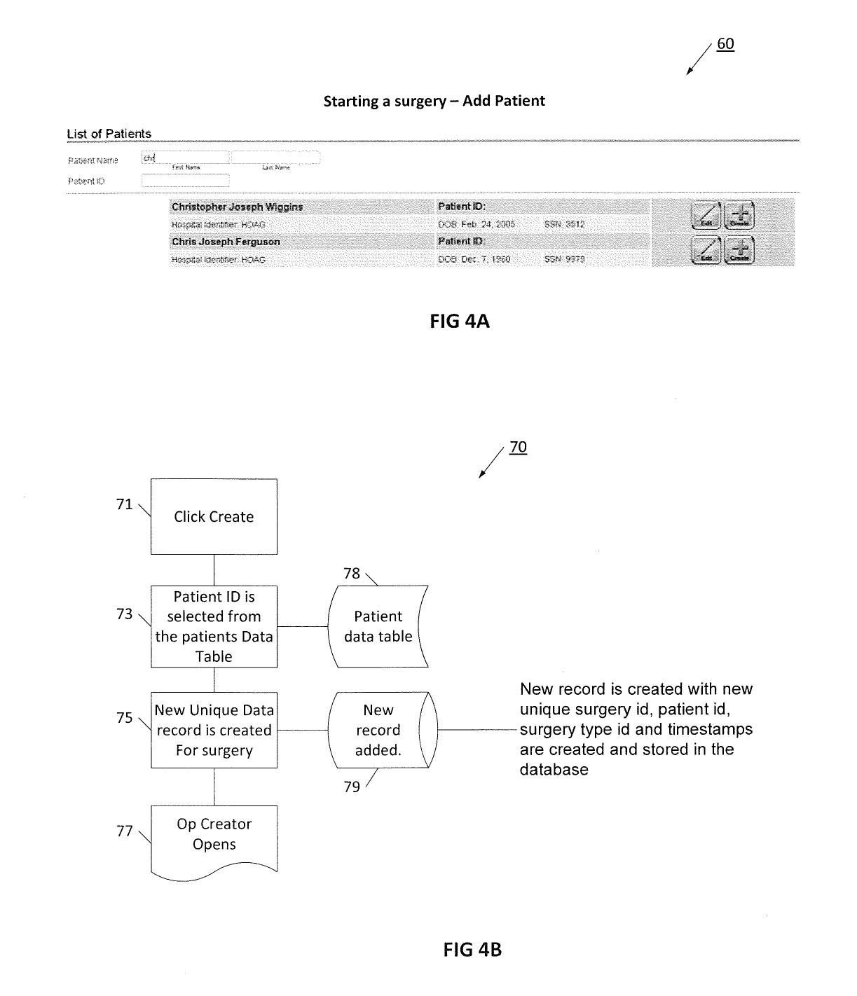

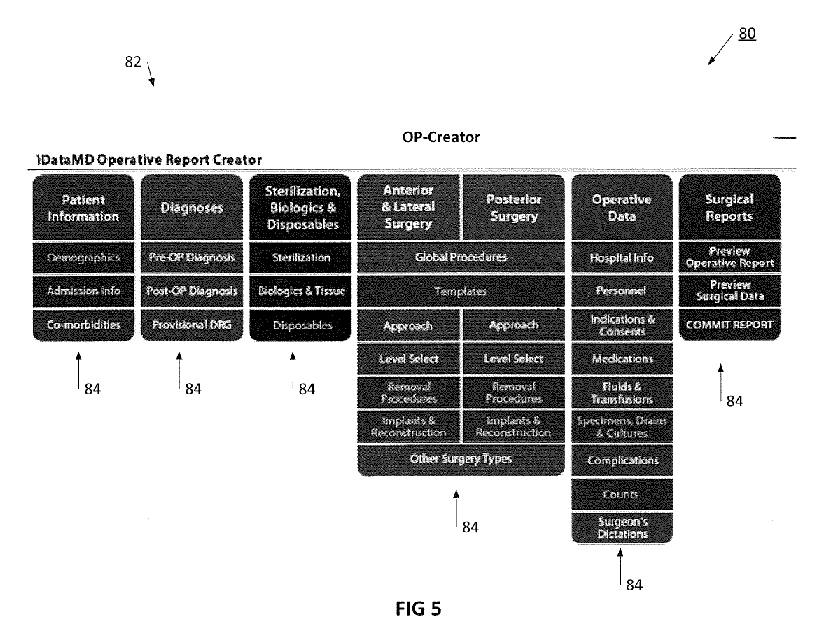

As illustrated in FIG. 4A and FIG. 4B, when starting a surgery, the surgery patient can be identified by name, ID, or other indicator. In one example, the system allows a search screen 60 for selection of an existing patient. It will be understood that certain information regarding a patient may be preexisting in the system, or may be accessible through automated access processes.

Flowchart 70 illustrates one process for creating a new surgery. Once the user selects to create a new surgery 71, the user is allowed to select a patient 73 using a patient ID, name, or other criteria. Typically, the patient information will pre-exist in a patient database 78 maintained or accessible by the medical provider. With the patient identified and the surgery type selected, a new data record 75 is generated for the specific surgery and locally stored 79. In this way, this new record holds information regarding the surgery type, patient information, and holds the information collected are captured during the surgery. As a consequence, this new record acts to temporarily maintain a complete record of the individual surgery, that allow the doctor to verify and confirm the information prior to the record being released into the wider medical data system. However, due to the systematic and near real time collection of surgical data, the process of verifying and confirming the surgery procedures and outcomes is highly automated and efficient. Often, a doctor or person in charge of the medical procedure generates a surgical report that provides detail regarding the surgical procedure. Advantageously, the surgical data system substantially automates the process for generating this surgical report, thereby enabling the doctor to verify and finalize his or her authorizations promptly upon completion of the surgery. Once the patient has been identified, the system allows for entry of data specific to that surgery, as illustrated in box 77.

As described with reference to the illustration of FIG. 5, the surgical data system enables real time collection for a surgery, and maintains the surgery information in a temporary record file prior to committing the data to a permanent operative report. In a typical process, the surgeon or medical person in charge of the medical procedure must verify and approve the surgical information, which is often done in the form of a surgical operative report. The surgical data system allows the doctor to incorporate the information collected in near real time during the surgery as well as have further patient, surgery, and outcome information included. In many cases, it will be more convenient or efficient for the doctor or other medical manager to enter some sections of the operative report prior to the surgery. For example, some patient information may be extracted from other medical records or input prior to the surgery. Also, the doctor may include a pre-operative diagnosis as well as make pre-selections for expected implants, biomaterials, parts, or tools to be used during surgery. Other parts of the operative report creator are reliant upon the real time data collected during the surgery or medical process. Advantageously, much of the real time surgical information is collected as graphical information associated with an anatomical representation of the surgery area. Other textural information or comments may also be collected. The operative report creator 80 also allows the doctor or medical manager to include post surgical information for the report. It will be appreciated that the particular items for selection on the operative report creator 80 will vary depending upon the type of medical procedure or surgery selected.

As illustrated in this FIG. 5, the surgical data input selections 82 allow for entry of patient information such as comorbidities to be attached to the patient record. Comorbidities are important to track as many insurance companies or governmental providers allow increased payments for patients having other indications such as diabetes. Although FIG. 5 shows morbidity information being input in the operative report creator, such information may be extracted from prior medical records or input by another prior to the surgery.

As illustrated in FIG. 5, the operative report creator is divided into over 20 major input sections 84. It will be appreciated that more or fewer input sections may be used depending upon the particular procedure performed. It will also be appreciated that the categories may be adjusted according to application-specific needs. Generally, the operative report generator input screen 82 allows input or confirmation of patient information such as comorbidities and general patient history. The diagnosis section allows for capture of the doctors pre-operative diagnosis, as well as input for post-operative plan. Obviously, the pre-operative diagnosis may be done prior to the surgery, while the post-operative diagnosis is something typically done after completion of the surgery. The system also allows the surgical plans to be modified in near real time during a medical procedure to reflect the actual condition of the patient. The system also allows input or verification of the sterilization history for parts and tools, as well as tracking for biologicals and implants. Some of this information may be input prior to the surgery, some collected real time during the surgery, and some verified or input after the surgery is complete.

The next section provides for review and supplementation of the near real time capture of surgical data. As will be described in more detail, the surgical data system allows for near real time capture of surgical procedures using an anatomical graphical representation. Using the functions 84, the doctor is able to review the collected information and confirm that the capture of data was complete and accurate. Other more textual operative data may also be collected, such as hospital and surgery room information, personnel performing the procedures, medications or other pharmaceuticals used during the surgery, any complications that arose during surgery, and even a transcript of surgeon or doctor comments. It will be appreciated that some of this information may be input prior to the surgery, some collected in real time during the surgery, and some added or supplemented during preparation of the operative report.

Once the doctor or medical manager is satisfied with the completeness and accuracy of the information in the temporary data file, the surgeon may print out and review and upper report, and then commit the report as a permanent record. When the report is committed, the temporary record is transferred in to the patient's permanent medical record, and no further changes can be made to this surgery report or information.

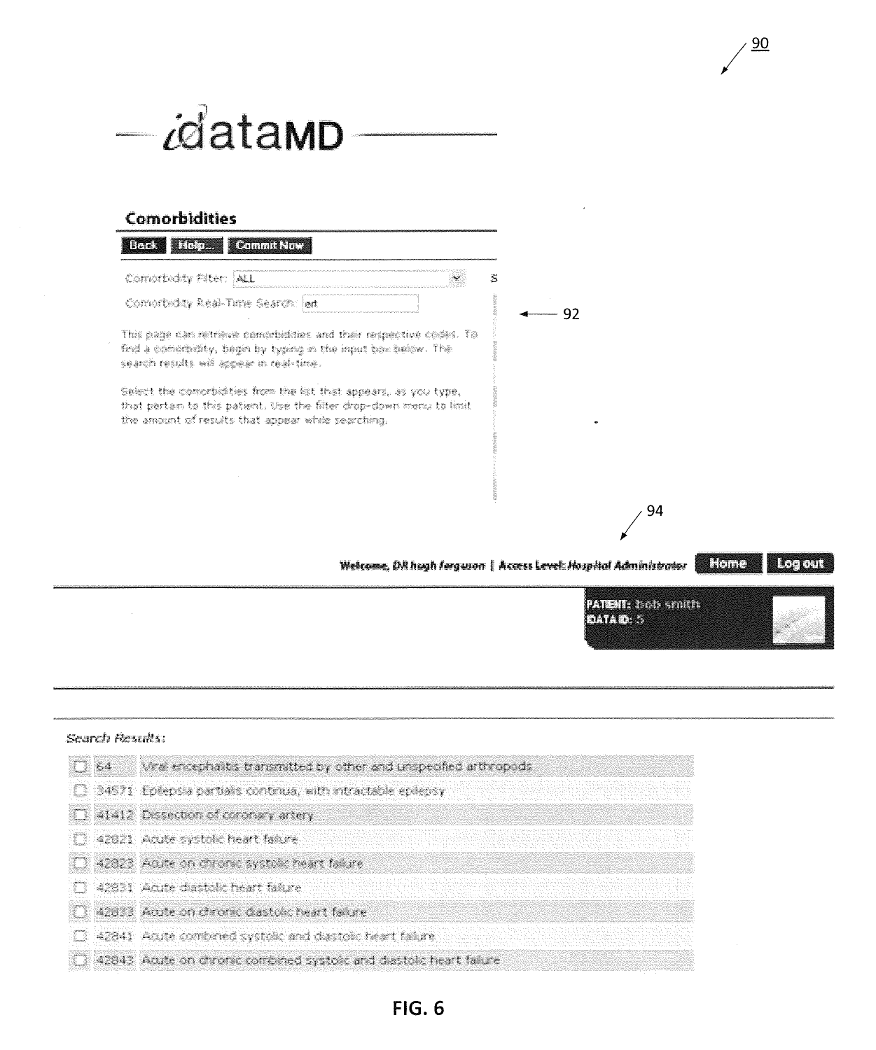

As described with reference to FIG. 6, an input screen 90 allows for input of input of specific comorbidity information 94, which is presented in a form for immediate use in billing and insurance purposes. By including industry codes, and restricting the operator to authorized comorbidities, more effective insurance and billing is facilitated. Due to the large number of standard comorbidities, a structured search screen 92 may be used to narrow the number of choices presented through input screen 94. The accurate collection and tracking of comorbidity information is important for patient care as well as proper billing for surgical services. The surgical data system has a database of known comorbidities, and provides an indexed, searchable database of comorbidities both by common description and by industry standard coding numbers. In this way, a doctor or nurse may search for and identify a comorbidity by the common medical name, and the surgical data system will automatically attach and track the particular insurance or governmental code required for billing.

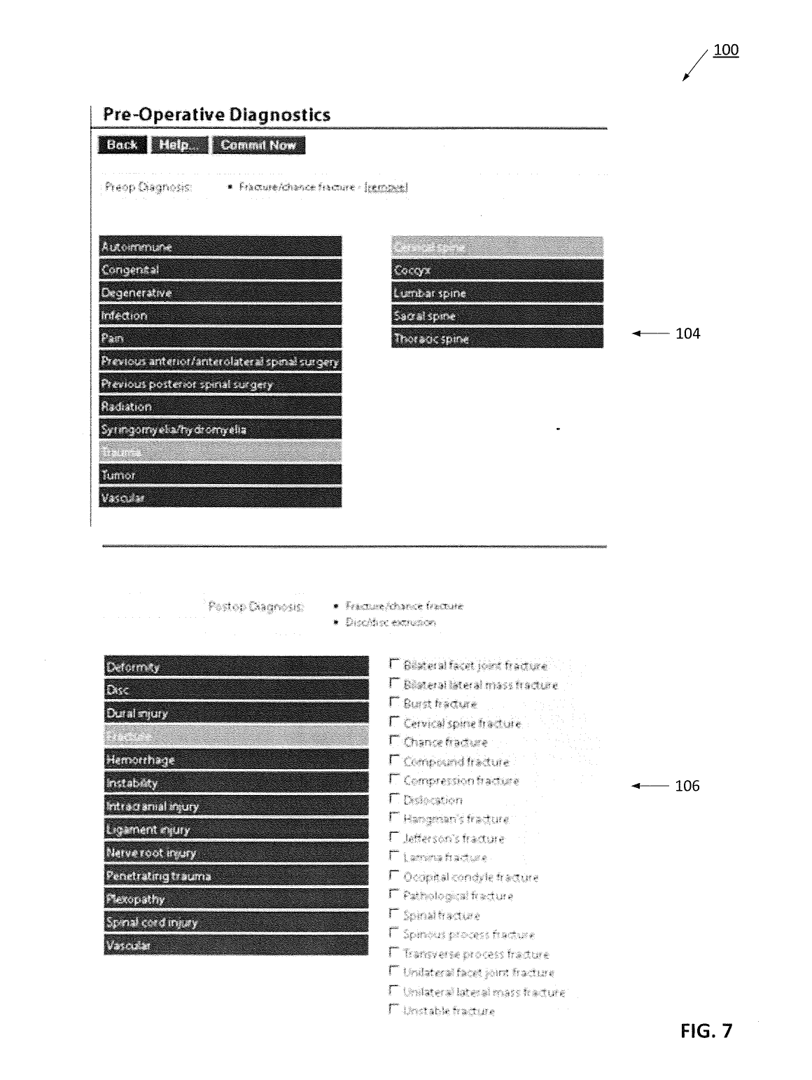

Referring now to FIG. 7, an input screen 100 allows for input of specific pre-operative 104 and post-operative 106 data. The surgical data system allows the doctor or medical manager to use a consistent interface and selection process for defining and capturing both the pre-operative diagnosis and the post-operative diagnosis. Advantageously, the surgical data system allows for both a pre-surgery injury of diagnosis, as well as near real time input of diagnosis during surgery. For example, once the surgeon begins the operation, if the surgeon makes further diagnoses, that diagnosis can be added in near real time during the surgery. Finally, after the surgery is complete, the surgeon or medical manager may add a post-operative diagnosis. Again, as the user interfaces are consistent, the adding, adjusting, and supplementing diagnoses is a simplified and automated process.

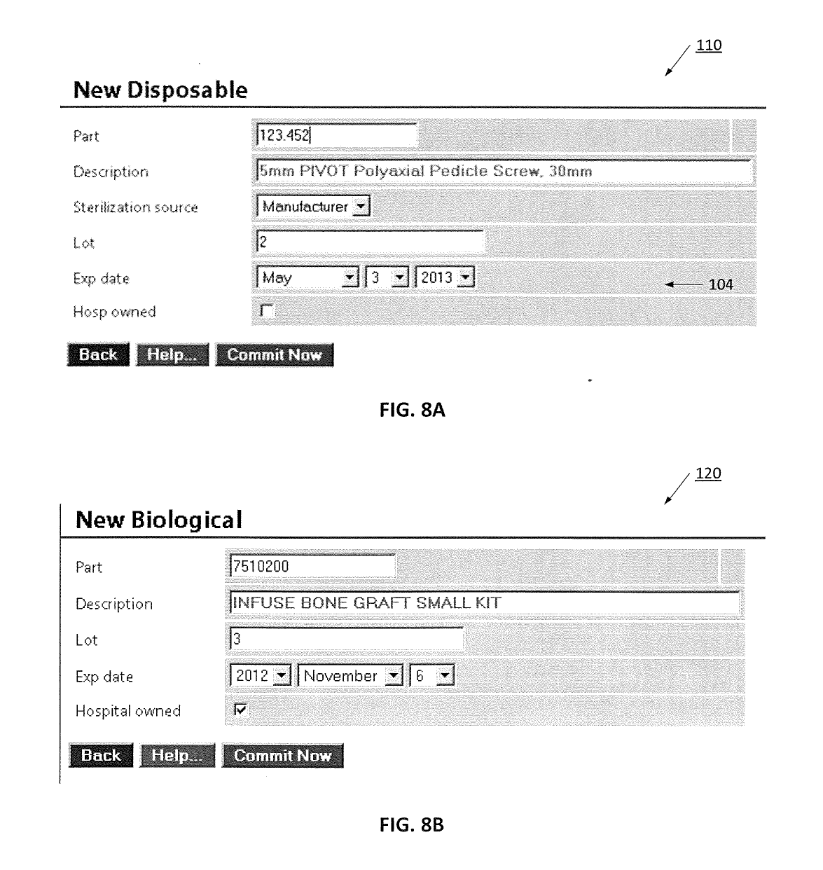

Referring now to FIGS. 8A and 8B, an input screen 110 allows for input of specific disposable information 100 or for biological information 120. The medical data system allows detailed tracking of disposables, parts or tools that are used during the surgery but that do not remain in the patient's body after surgery. It is important that the sterilization history for every disposable be understood and verified before use, and a history of sterilization maintained in the patient's record. Some of the information regarding sterilization may be entered prior to the surgery, but since the particular tools and disposables being used during surgery often are not known until the surgery is underway, the surgical data system enables a simple interface for identifying new disposables and confirming sterilization information. Importantly, if a disposable is selected for use, its sterilization history is immediately presented to those in the surgical room, and an improperly sterilized disposable can be identified and excluded from use. In a similar way, biologicals are tracked and verified prior to use in the surgery. For example, some biologicals have an expiration date, which is presented to those in the surgery room prior to the biological being used. An expired or questionable biological can be excluded.

The surgical data system may be pre-populated with expected disposables and biologicals. Alternatively, the surgical data system has an interface to the hospital or medical center database and the information regarding disposables, sterilization, and biologicals may be retrieved from existing database information. In this way, the surgical team uses only items authorized by the hospital and administrative team. Using any process or part that is not part of the pre-authorized database may result in a warning to the surgery and administrative teams, and the unauthorized action can be investigated, reducing the opportunity for fraud or mistakes.

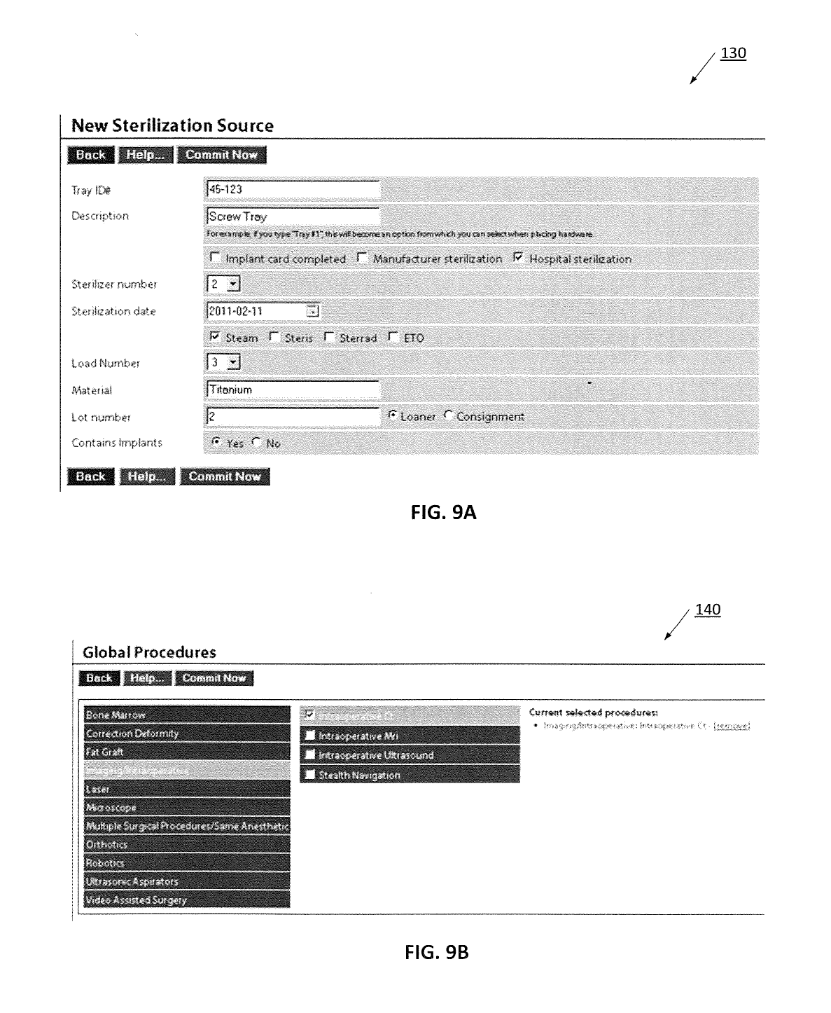

Sterilization information is maintained, tracked, and verified down to the individual part as illustrated in the input screen 130 of FIG. 9A. Often, sterilization is done on a tray-by-tray basis, so sterilization also includes information on the particular tray to be used. As illustrated in FIG. 9A, detailed information as to the type of part, the sterilizer used, and the date and type of sterilization process may be maintained. Also, an expiration date for sterilization may be provided.

The surgical data system also contemplates collecting global procedures data using input screen 140 as illustrated in FIG. 9B, which are medical or surgical procedures that are not specific to a particular surgery or patient location. It will be appreciated that many other types of global procedures are contemplated.

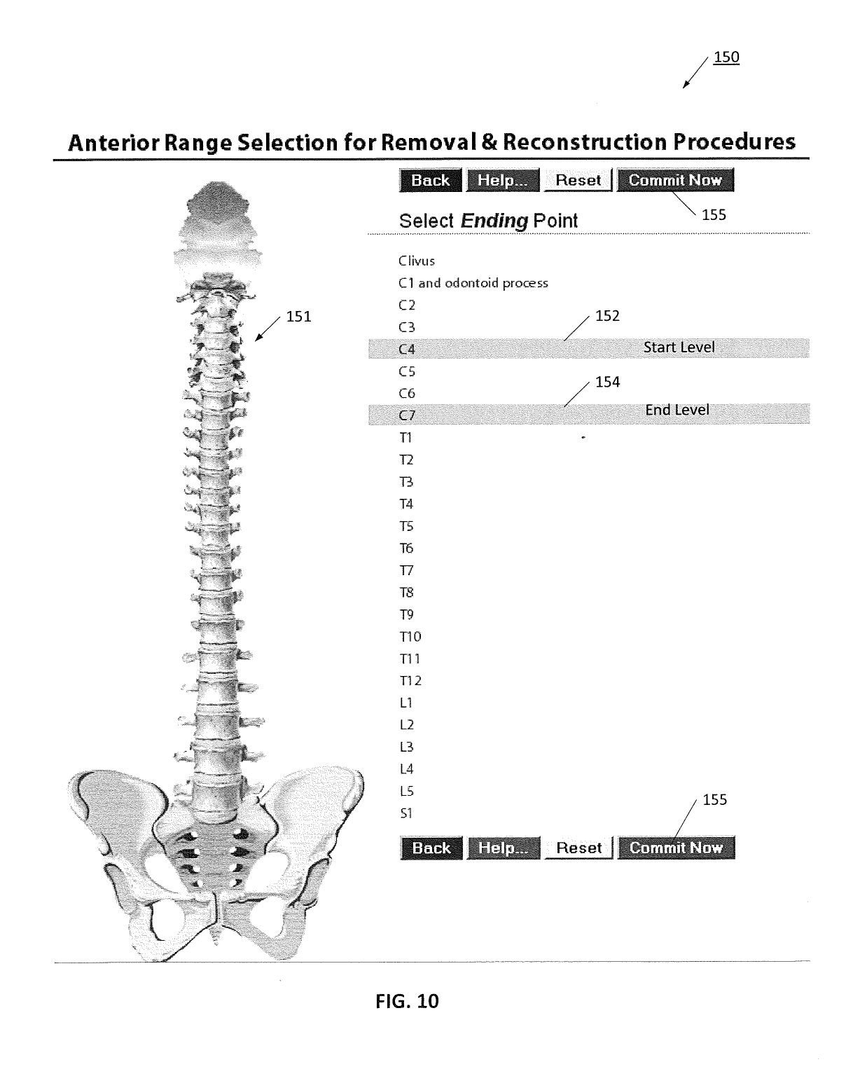

Upon selecting a surgery type, the surgical data system accesses a surgery type database and retrieves anatomical representations for that selected surgery type. As illustrated in FIG. 10, and operator has selected to do a spinal procedure. In some cases, a surgery type may be specific to the actual surgery site, while in other cases the selected surgery type may require multiple levels of selection prior to finding the precise anatomical representation for the surgery at hand. For example, FIG. 10 shows an initial screen 150 after a spinal surgery type has been selected. In the case of a spinal surgery type, and anatomical representation of an entire human spine 151 is first displayed. Medical personnel then select which spinal levels the operation is to be performed, using either the graphical 151 or tabular 152, 154 inputs. Once the operator has selected the proper range, he or she commits 155 the process and an anatomical representation of the selected surgical site is then presented to the surgical team.

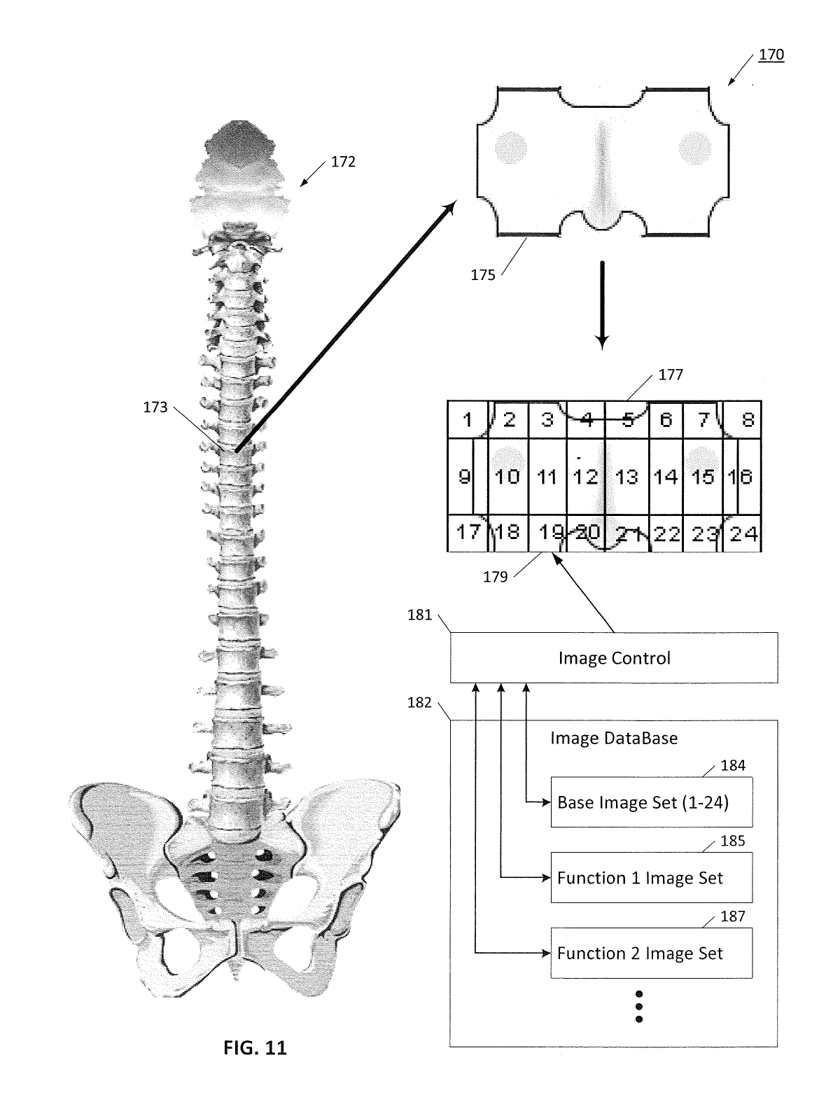

Referring now to FIG. 11, anatomical representation 170 is illustrated. The anatomical representation 170 includes a full spinal display 172 comprising individual levels, such as level 173. In one example, each spinal level is individually generated and then arranged with other illustrations of spinal levels to build the final spinal representation 172. As will be seen later, this anatomical representation of the spine becomes a base graphical layer for enabling real time capture of surgical or medical procedure data.

In one example, each spinal level, such as level 175, comprises a set of individual graphic elements as illustrated in block 177. Here, each individual spinal level is divided into 24 separate graphical images, such as image block 179. It will be appreciated that the number of image blocks used, their size, and their relative size, can be selected according to application specific requirements. Here, as more detailed information is typically collected at the top and bottom of the spinal level, the image blocks at the top and bottom are made smaller than those in the middle. As illustrated in the flow chart, an image control process 181 determines which specific graphic is displayed in the anatomical representation. The image control 181 is able to select a specific image from an image database. For each block, such as block 179, a series of available images may be stored. In one example, the image database 182 holds a base image 184 for each of the 24 image blocks. Additionally, there may be images specific to particular procedures as shown in blocks 185 and 187. For example, a block may be prepared in advance and stored in the database that is indicative of a bone removal procedure. In this way, if bone were removed, for example, at image block 179, the base image of block 184 would be replaced by the image 185 or 187 containing a crosshatch or other indicator of removal. It will be appreciated that not all image blocks will have alternative function images.

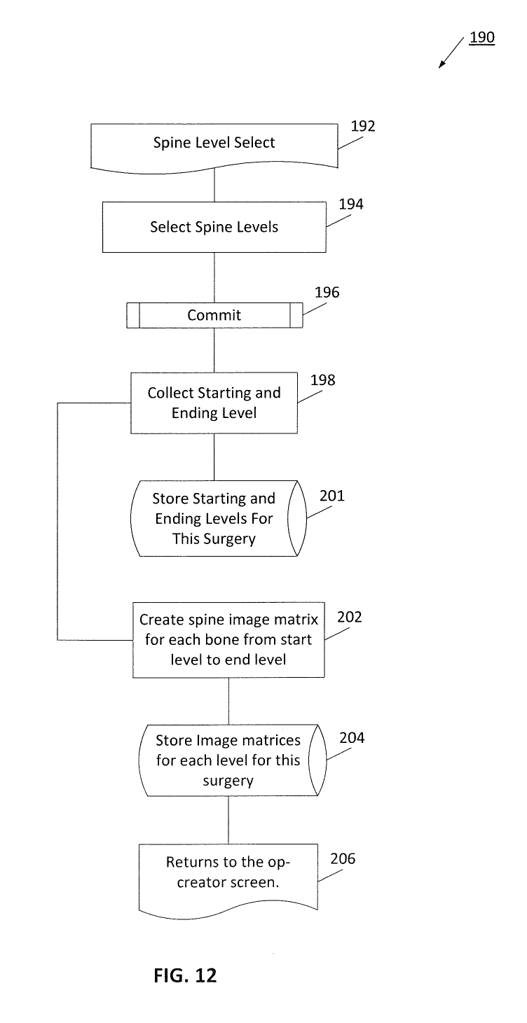

Referring now to FIG. 12, the surgical data system is constructed such that the anatomical representation for the specific spine section is built only after the specific surgery site has been selected. In particular, the medical operator selected a start point and end point on the spine and then activated the commit button. For every level selected, the anatomical representation for each level is extracted from an existing database and the anatomical representation for the full surgical range is created. This is saved and stored along with the temporary surgery information for use and annotation during the surgery.

As illustrated in FIG. 12, the medical data system has a spine select process 190 that is used for displaying the particular spine levels for a surgery 192. In doing so, the operator selects a range of spine levels to operate on as shown in block 194. Typically, the user would select a starting level and a stopping level. With the start and stop positions identified, the user hits a commit button 196, which then drives the process of displaying the specific anatomical representation of the surgical area. More particularly, the controlling software collects the starting and ending level at 198 and then extracts images from a database to create the representation of every level, with each level comprising an image matrix as illustrated in box 202. The information regarding starting and stopping levels is also stored as shown in block 201, for future use in preparing the operative report. In a similar manner, the image of the anatomical representation is also stored as shown in block 204, as it will also become part of the permanent operative report. Once the specific surgical areas are displayed and stored in the system, the user is then returned for further input as shown in block 205

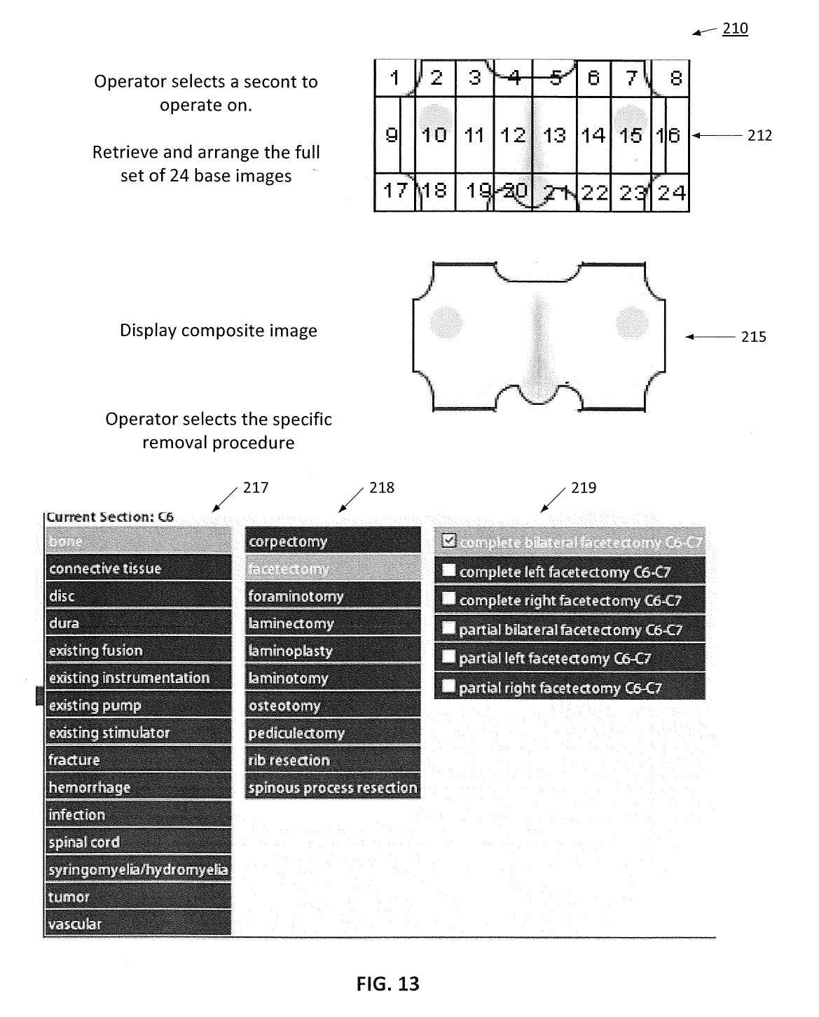

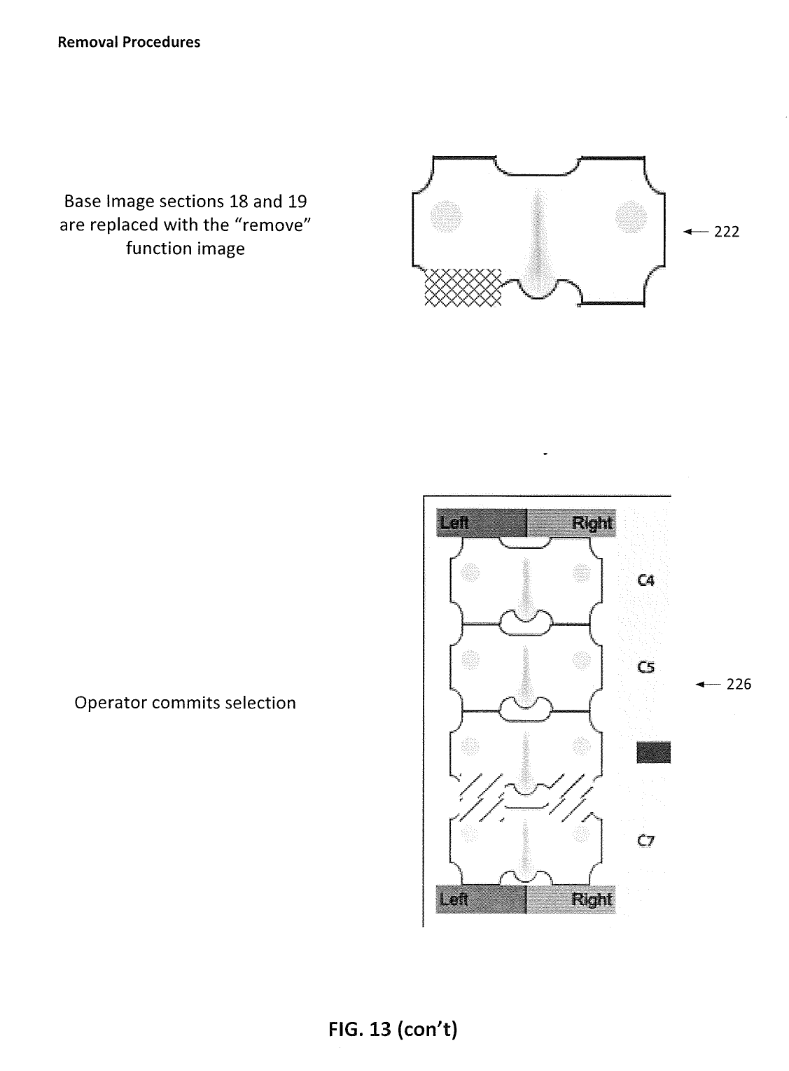

Referring now to FIG. 13, an example 210 for collecting information regarding tissue and bone removal is illustrated. As described with reference to FIG. 12, each spine level 215 is divided into 24 individual portions 212. It will be understood that other numbers of portions may be used depending on the surgery type. Accordingly, as a surgeon removes tissue, an operator uses the tab boxes 217, 218 and 219, as illustrated in FIG. 13, to specify the spine level that is being worked on, and then the specific type of removal or procedure performed on that level. The system selects the appropriate graphical image to represent the specific surgical task selected, and replaces one or more of the 24 portions to annotate what the surgeon did. As an example, the operator has selected a specific bone removal procedure using the tab boxes 219. In response, the system retrieves “removal” function images from the database for portions 18 and 19, and replaces the base anatomical image with crosshatch graphics, as illustrated at 222. More particularly, the entire anatomical representation is updated to show the removed sections as shown at 226. In this way, the operator and the surgeon can visually confirm that the proper procedure has been captured in near real time with the performance of the procedure. It will be understood that other combinations of graphical and tabular selection, manipulation processes, and presentations can be used.

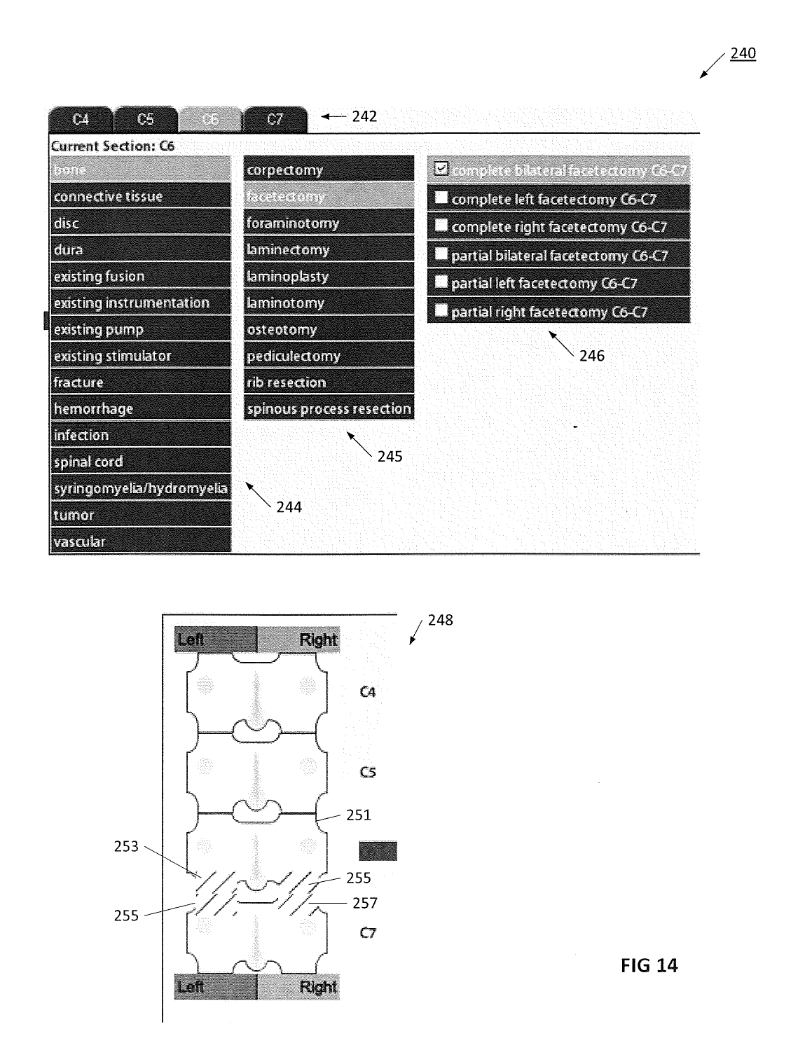

FIG. 14 shows an anatomical representation 248 of the C4 to C7 spinal section. The anatomical representation is associated with a tabular display 240 identifying the available procedures for each spine level. In this way, information may be provided graphically on the anatomical representation, and additional detail may be collected using the drop-down box selections. Indeed, a relationship exists between items selected on the tabular drop-down boxes and graphical display. For example, as illustrated, the surgeon has indicated that there will be a particular removal procedure done in the C6 section of the spine, as captured in area 242. Upon selection of C6, the anatomical representation may activate a removal layer that enables the computer operator to graphically indicate the specific areas of bone removed. Alternatively, the graphical representation may be automatically updated responsive to the specific removal selected in the drop down boxes, 244, 245, and 246. The boxes are arranged in a hierarchical arrangement that allows the operator to first select the broad type of procedure that is being performed 244, and then make more granular selections using boxes 245 and 246. This acts to simplify the interface to the user, and to keep the number of specific procedures to a usable level. It will be understood that more or fewer levels can be used in the hierarchical presentation.

In this graphical display the particular type of removal is illustrated as a crosshatch. In a similar manner, reconstruction, or placement of implants may be indicated in whole or in part in a tabular form, which then may activate the appropriate layer for that function, thereby enabling a graphical positioning and representation of the surgical procedure. When selecting a removal procedure, the system searches an image-change database and if the identifier of the selected procedure matches an identifier in the image-change database, bone images in the matrix with be replaced with images that represent the actual bone removal.

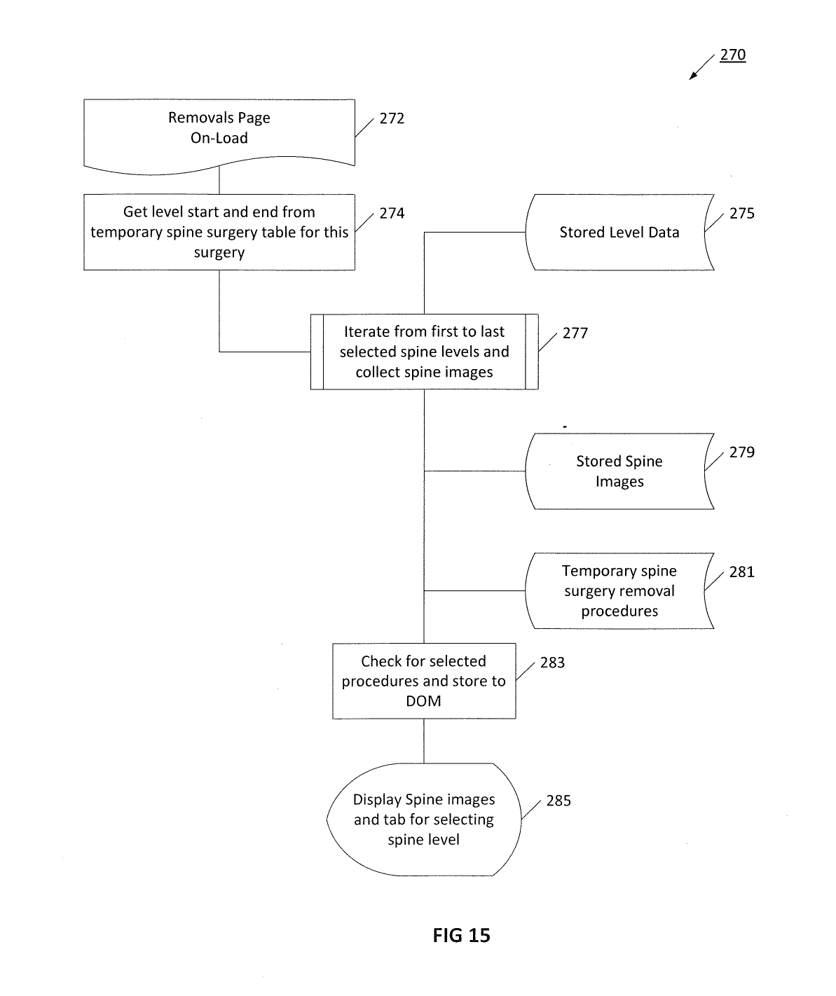

Referring now to FIG. 15, a flow chart 270 is illustrated generally describing the initial aspects of presenting and using a removal procedure. A removal process 272 starts by having the operator define a spinal level start position and a spinal level end position using either tabular input or a graphical selection from a presented anatomical representation as shown in block 274. In one example of the medical data system, the individual spine levels are separate graphical structures, and each one is extracted from a graphical database and presented sequentially as illustrated in box 277. As previously described, the graphical images for each level may be stored as sets of graphical images as described in block 279. Other level data may be stored and used as shown in block 275. This information may be for example information regarding prior surgeries, prior removals, or prior hardware already existing in the selected areas. As illustrated in box 281, the present surgery may be a continuation of a prior spinal surgery which was either completed or not completed. Accordingly, as shown in box 281, information regarding the current status of the surgery may be temporarily saved. The graphical information for all of the selected spinal levels, along with any temporary procedure information, is retrieved from the data storage area and placed in a local memory 283 for more efficient and faster access. To name a specific example, the local memory storage may be in the form of a DOM for an HTML supported system. The entire selected spinal area and existing procedure information is thereby efficiently displayable to personnel in the operating room as shown in block 285. Further, due to the local storage in readily accessible memory, the system remains highly responsive to commands and inputs from the operator. By offloading computational operations from the server to the client side, the overall system is enabled to operate more efficiently, to be more scalable, and to enable remote and distance access.

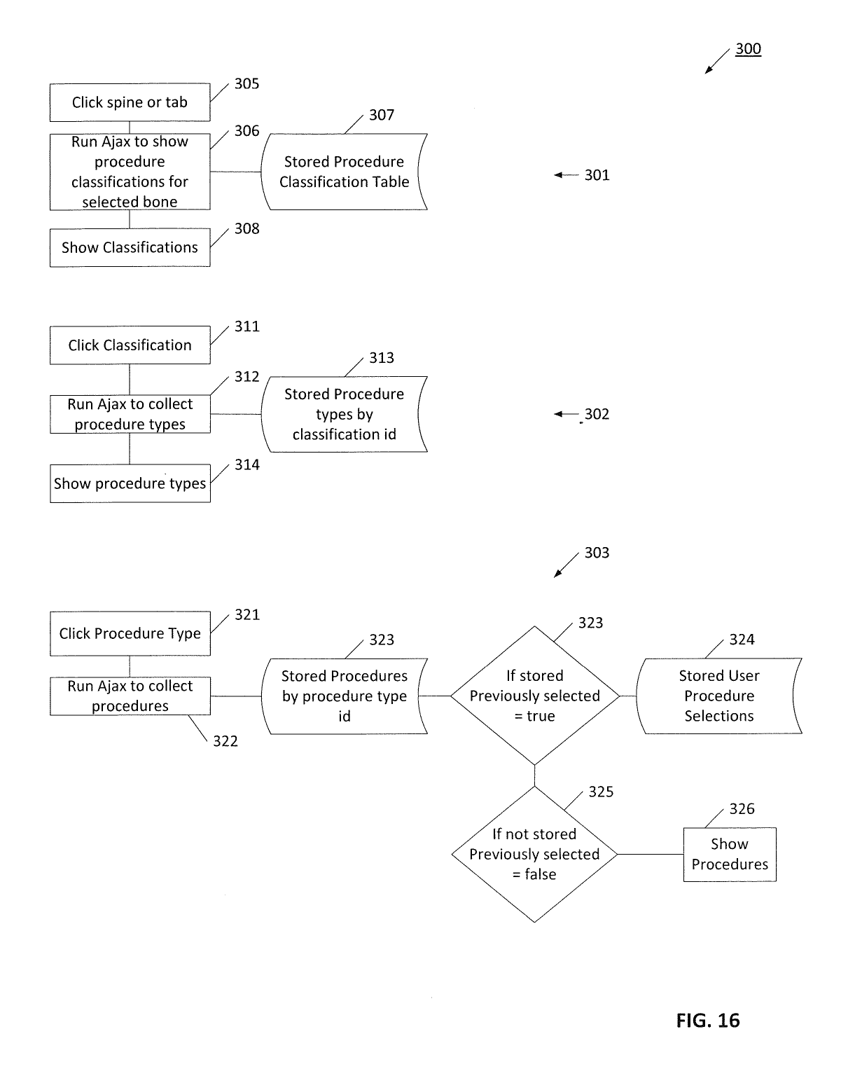

Referring now to FIG. 16, a series of flow charts 300 are used to describe a tabular process for performing spinal level removal. FIG. 16 is described with reference to the tabular display illustrated in FIG. 14. As shown in FIG. 14, the list of spinal levels selected for surgery is shown across the top of the display 242. In this specific case, sections C4 through C7 have been selected for the present surgery. Referring now to chart 301, an operator clicks on the specific section or level 305 for performing a medical procedure such as a removal. The system then retrieves from a database 307 available classifications, displays a set of classifications for that bone as shown in blocks 306 and 308. In FIG. 14, the operator has selected the level C6 which causes the medical data system to display a list of classifications 244. As shown in chart 302, the operator then selects one of the available presented classifications 311. The medical data system then operates to retrieve from a database 313 and present the list of available procedure types for that specific classification as shown in blocks 312 and 314.

As illustrated in FIG. 14, the operator selected bone, which brought up the procedure types 245 for that specific classification. As illustrated in chart 303, now the operator selects one of the particular procedure types as shown on block 321 and the system retrieves and presents the specific procedures available for the selected procedure type as shown in blocks 322 and 323. Here, the operator has selected facetectomy, which has then brought up the specific procedure list 246. The system may then check to see if this specific procedure has been performed before as shown in blocks 323 and 325. If this is the first time the procedure has been done, then the removal procedure is indicated graphically on the display as shown in block 326. As described earlier, this entails replacing one or more of the specific image graphic blocks with an indication of bone removal. If this is a continuation of an operation as indicated in block 324, then the system uses the existing procedure data already stored.

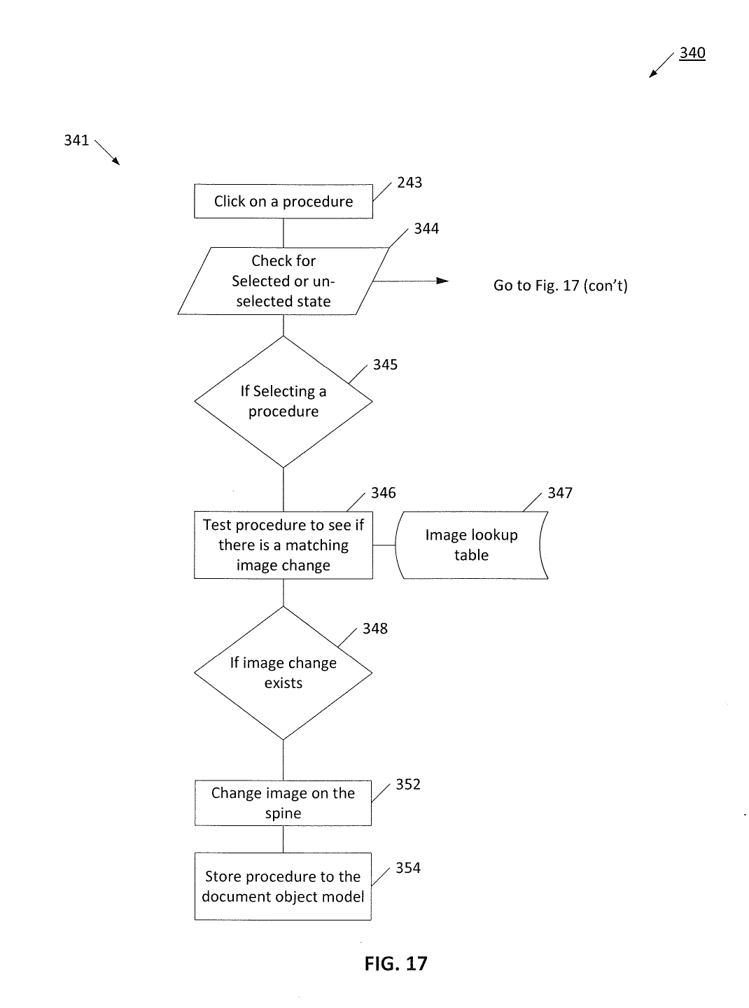

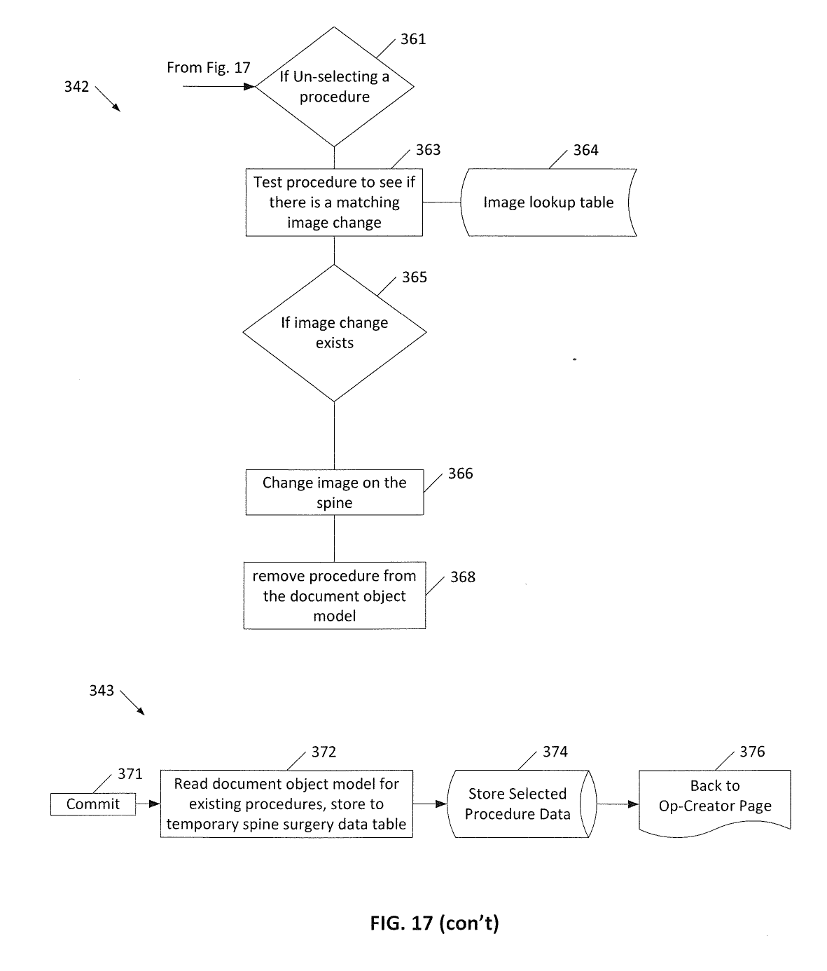

Referring now to FIG. 17, a flow chart 340 is illustrated showing actions the medical data system takes during a removal procedure. Section 341 of the flow chart shows that a user clicks or selects a particular procedure 343. It will be understood that this selection may be made graphically, or may be by selecting check boxes or other more textual or tabular processes. The system checks in box 344 to see if the operator is selecting to perform a procedure or unselecting a previously selected procedure. If the operator is selecting a procedure as shown in box 345, then the system proceeds to check if that specific procedure requires an image change to the anatomical representation as shown in block 346. In doing so, the medical data system may refer to an image table 347 to determine which, if any, image change is required for that selected procedure. If the selected procedure requires an image change as shown in block 348, then that image is retrieved from storage and displayed on the anatomical representation as shown on block 352. The medical data system also stores the new procedure in its local memory system as shown in block 354.

In the case where the operator has unselected a procedure as shown in flow chart sections 342 and 361, then the medical data system proceeds to determine if that previously selected procedure required an image change as shown in block 363. As before, the system will use an image look-up table 364 to determine which procedures have an associated image change. If an image change was done on that prior procedure, as shown on block 365, then the original anatomical image portion is retrieved from the database and displayed back on the anatomical representation as shown on block 366. Whether or not an image change is required, however, the system will update its local memory to indicate that that procedure was not actually performed, as shown on block 368.

The medical data system also has a commit process, 343. During use, the medical data system retains additions, deletions, and changes made to by the operator in a local memory. By maintaining a local memory version of the putative changes, an operator has some flexibility in correcting inadvertent inputs to the system; for example, as described above in the unselecting process. However, once the operator has confirmed that the data input and the anatomical representation are correct, then the operator may select a commit function as shown on block 371. Once the commit button has been hit, then the system takes the information from a local temporary memory storage, 372, and transfers that to a permanent data storage facility 374. Once this procedure has been completed by committing it to long-term storage, then the operator is taken back to begin the next procedure or data input as shown in block 376.

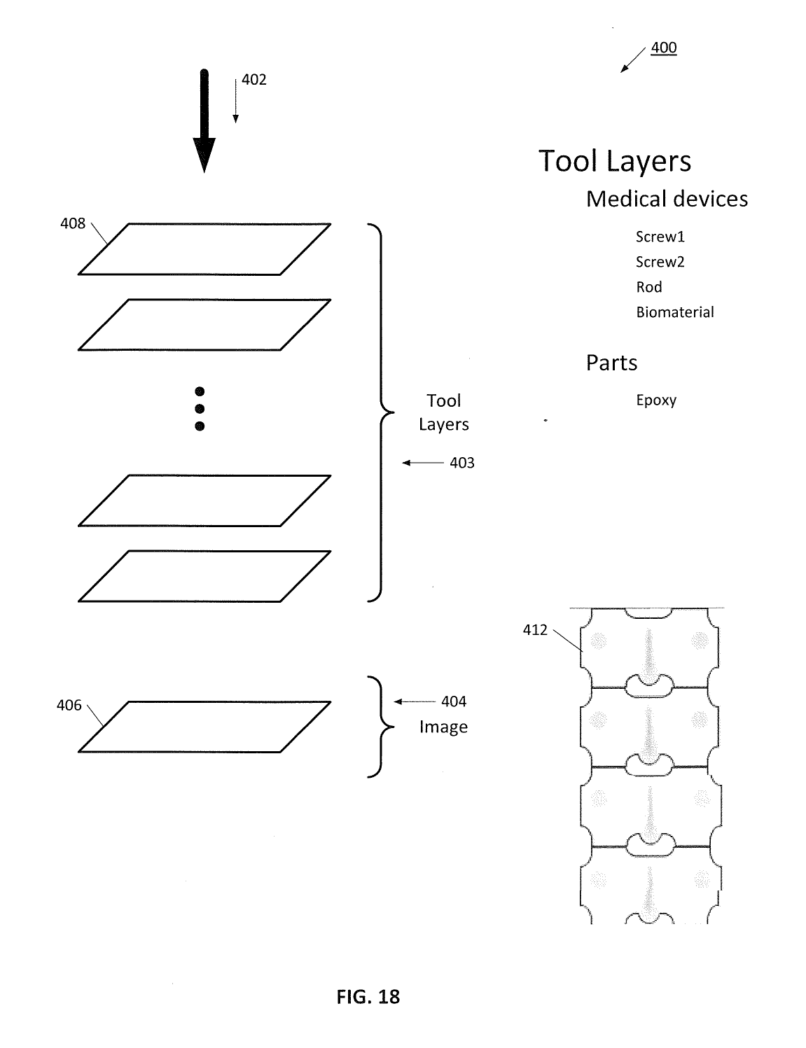

Referring now to FIG. 18, a process 400 for collecting real time information regarding reconstructive surgery is illustrated. Reconstructive surgery may include the addition of biological materials, hardware, electronics, or other devices or implants placed into the human body. For example, the spinal surgery discussed in this portion of the disclosure may require the addition of screws, rods, and other hardware supporting devices, as well as bone material or other biomaterials. To allow for real time, accurate, and verifiable input of such reconstructive information, the medical data system provides graphical assistance by placing a set of tool layers, 403 above an image data 404. In one example, image data 404 is the anatomical representation 412 discussed previously. Typically, this anatomical representation 412 will be the bottom-most layer 406 in a stack of image layers. Sitting above the image base layer 406 are tool layers 403. Each tool layer represents a particular tool. For example, one layer, such as layer 408, may provide for the placement of spinal screws. Another layer may be associated with placement of rods, while another layer may enable the data collection regarding using biomaterials or adhesives. As will be described in more detail later, the tool layers 403 are positioned in a vertical arrangement that enables an operator to see an accurate representation of the results of the surgery by viewing the stack layer from direction 402. It will be appreciated that the particular ordering of the tool layers may adjust during operation of the medical data system. For example, to manipulate or use a particular tool layer, it may be brought to the top of the stack for more precise input operations. However, when that particular tool has been completed, the tool layer may move to a lower location more representative of how a doctor or other medical personnel would actually view that tool positioned within the body.

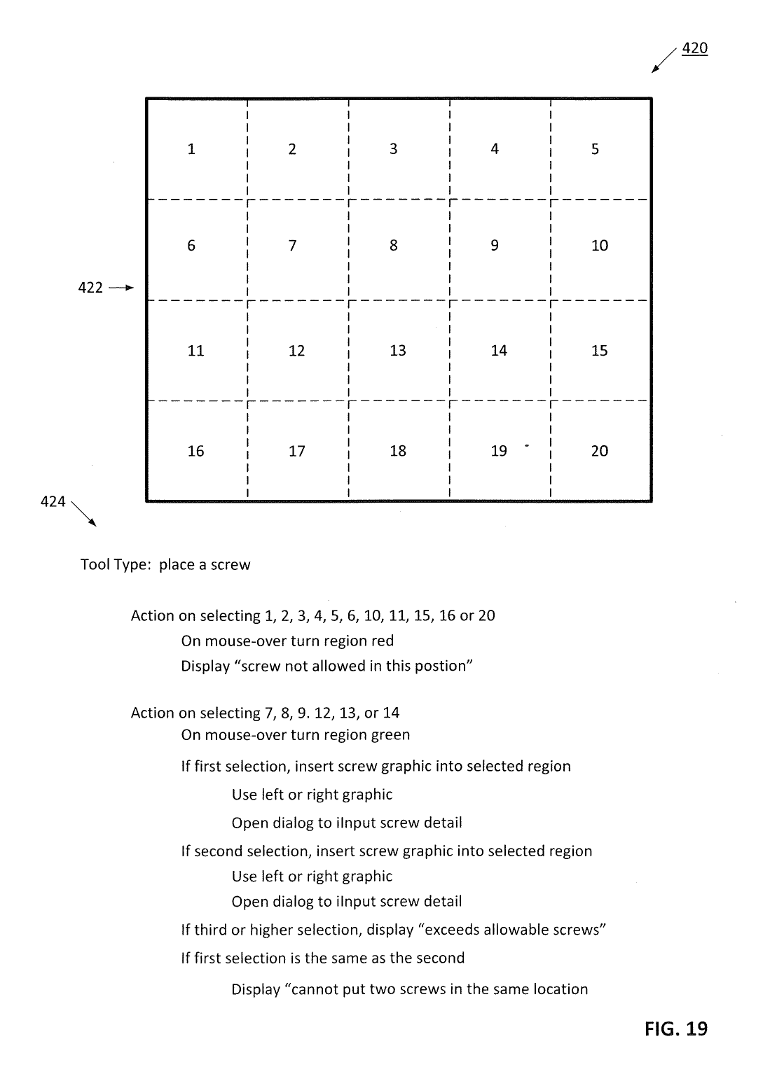

Referring now to FIG. 19, a particular tool layer 420, is illustrated. Typically, a tool layer is divided into a set of action areas. In particular tool layer 422 the layer has been divided into 20 equally sized action areas. It will be understood that more or fewer areas may be set, and the sizes may vary depending on tool requirements. Importantly, a set of rules, most often implemented as a set of software code, is associated with each tool layer. In a particular example, a set of rules 424 is applied to layer 420 for the placement of a metal spinal screw. As illustrated in rules 424, individual action areas may be assigned certain constraints, rights and capabilities. For example, the rules 424 show that the upper and side rows of action areas are not available for placing a screw. For example, if the operator uses a mouse or other graphical device to position a cursor in any one of those boxes, the box will provide an alert that indicates that no screw is available to be placed in that box. In this way, the intelligent tool layer 420 can restrict the operator from accidentally or fraudulently placing a screw where one should not be placed. In a similar manner, rule 424 indicates that selected interior action areas are available to have a spinal screw. Upon rolling over these action areas, the area would turn green. If the operator chooses to place a screw in one of the available boxes, a dialog box will appear that allows the operator to make specific choices as to the type of screw to be inserted. Once the screw has been confirmed to be positioned, then a graphic image is extracted from the database and placed in the proper action area. In this way, the tool layer shows a graphical representation of placement of hardware and biometrics.

Advantageously, as illustrated in rule 424, the system can also be intelligently designed so that the operator cannot inadvertently or fraudulently place more screws than allowable into a particular area. For example, the system may be set up that no more than one screw could be placed in each of the action areas. If the operator attempts to put a second screw in a box, the system will not allow that to happen and provide a warning. The system can also be defined such that the entire layer itself 420 can be set for a maximum number of screws. For example, as illustrated in rule 424, the maximum number of total screws allowed is 2. In this way, if an operator tries to insert a third screw into a spinal level, either inadvertently or fraudulently, then the system will provide a warning.

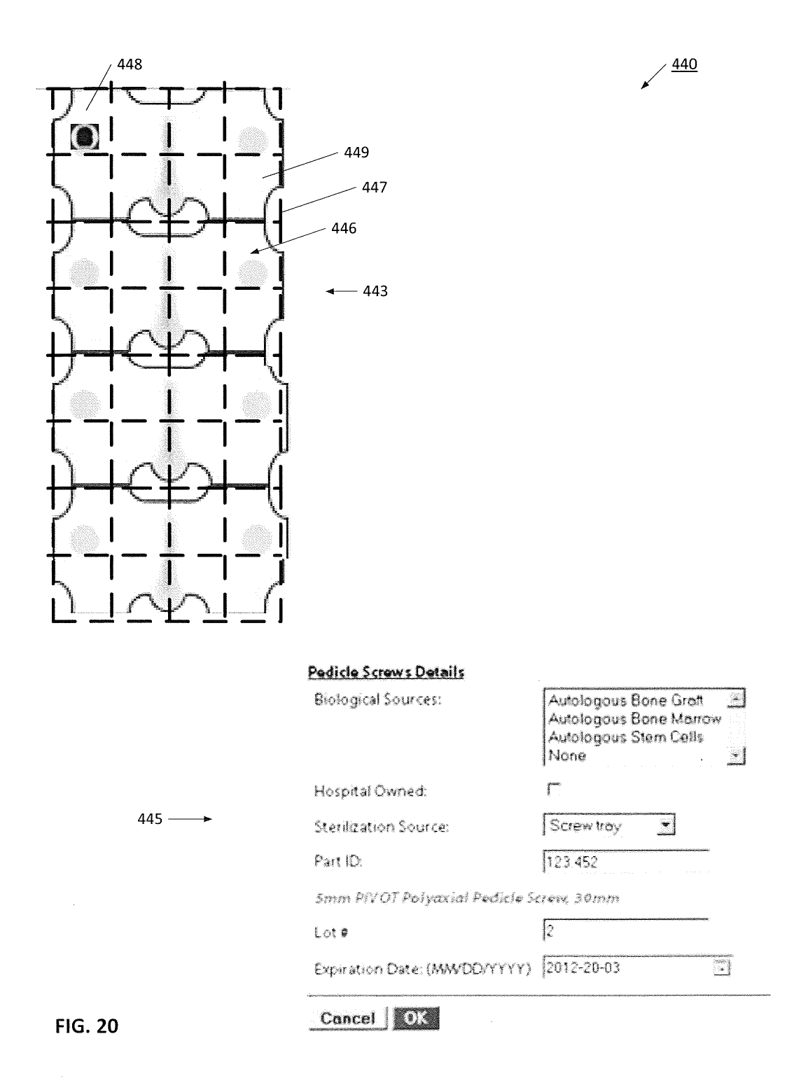

Referring now to FIG. 20, an anatomical representation 443 is illustrated for a reconstructive system 440. The reconstructive system 440 has an base image 446 for the anatomical representation 443 as described earlier. Here, the anatomical representation shows four levels for a spinal surgery. If any removals had been performed, they would be indicated with a cross-hatching on the anatomical representation 443. Since none exist, this particular surgery has not had any removal procedure. Overlaying each level is a matrix of action boxes 447. Each action box, such as action box 449, may allow for particular rules for that function, and may have alternative graphics that can be applied upon the performance of certain procedures. In one example, an operator uses the anatomical representation 443 to make a selection into box 448. In this example, action box 448 is associated with a layer for inputting screws. Box 448 has also been authorized for a screw, so upon the operator selecting box 448, a drop-down box 445, appears. Drop-down box 445 allows the operator to select the particular piece of hardware screw that the surgeon is using. The drop-down box 445 further allows identifications of the particular screw tray, as well as information regarding the sterilization of that screw. Accordingly, a complete historical record of that hardware screw is maintained by the system. Once the operator has selected the particular screw from 445 and selected the OK button, then the system performs a rule check to determine that that screw does not exceed the authorized number of screws for that action area or for that layer in general. It will be understood that other types of authorization, quality and fraud checks may be made. Once the screw has been approved for use in that action area, a graphical image of the screw is retrieved and placed in that action area as shown in block 448.

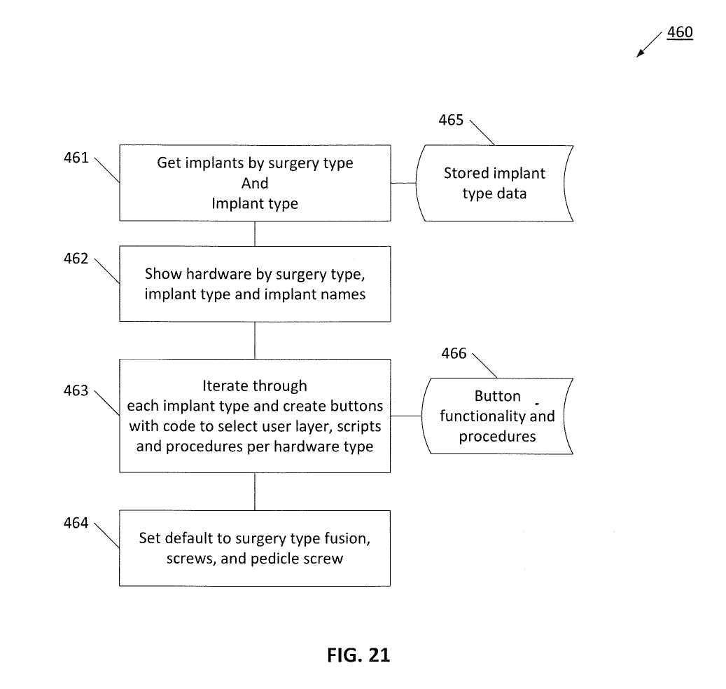

Referring now to FIG. 21, a method 460 is illustrated for associating specific hardware implants with a medical procedure, such as a surgery. As shown in block 461, a database 465 contains specific information regarding available hardware implants. For example, the database 465 may contain detailed information regarding screws, rods, hardware connectors, biomaterials, or adhesives. Once an operator has selected the particular surgery type, and indicated that reconstructive surgery will use a particular type of implant, such as a screw, the available hardware is then displayed to the operator as shown in block 462. In particular, each type of hardware implant may be presented to the user with functionality selections limited to the particular type of reconstruction being performed as shown in block 463. For example, a database 466 may include the specific functionality for each piece of hardware, which may be particularized to particular surgery types or reconstruction processes. For example, a particular screw may have a different set of rules associated with it, depending upon which particular surgery type or reconstructive procedure that has been selected. Once the functional buttons have been defined, the input screen is presented to the user, generally in the form as shown in input screen 445 of FIG. 20. In this way, the input screen 445 has the available defaults predefined as illustrated in box 464.

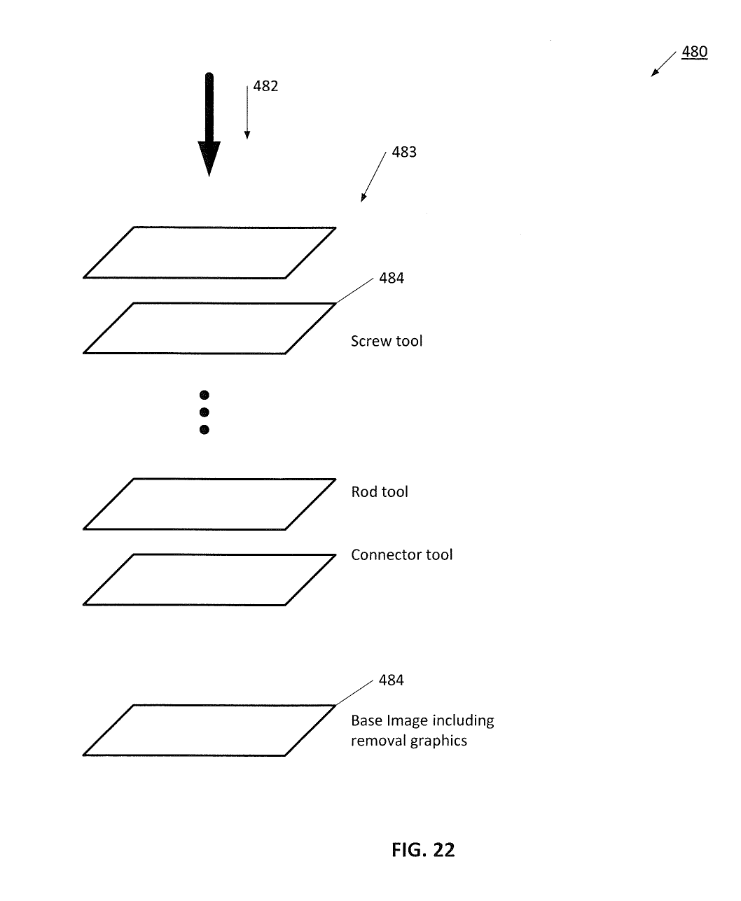

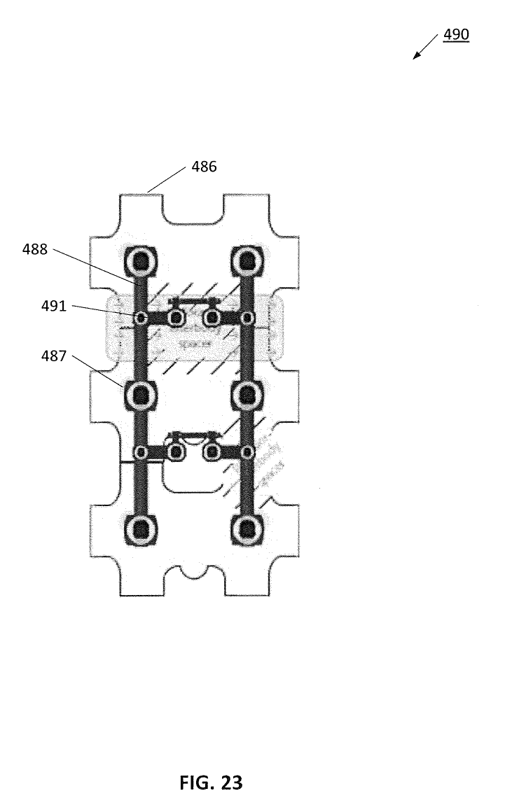

Referring now to FIG. 22, another method 480 for displaying reconstruction of a spinal surgery is illustrated. Display 480 has a base image 484, which includes any removal graphics. A set of vertically ordered layers 483, initially transparent, are positioned above the base image 484 in a viewing orientation 482. The stack of layers 483 contains several individual layers such as layer 485. For example, a layer may be used to intelligently place screws, another layer may be used to place rods, and another layer may be used to set connectors or other hardware. It will be appreciated that many other types of tool layers may be used. Also, it will be understood that the vertical arrangements of layers 483 may be adjusted such that, from viewing angle 482, the image representation is similar to what a surgeon sees when viewing the surgical area. For example, when viewed from viewing angle 482, screws will be most visible as a top layer, which may set on top of rods, which may set on top of connectors or other hardware. As illustrated in representation 490 of FIG. 23, three individual spinal levels 488 are shown, and the cross hatched removal area is illustrated on the base image. Both the anatomical representation, including the removal indicators, are part of the base image 484. When viewed from viewing angle 482, the screw images of the screw layer appear as screws 487 on the anatomical representation 486 of FIG. 23. Positioned just below the screws are rods 488, and below them are the connectors 491. It will be understood that the particular vertical arrangement of the layers may be adjusted; for example, by having the layer which is active moved to the top for better and more accurate visibility.

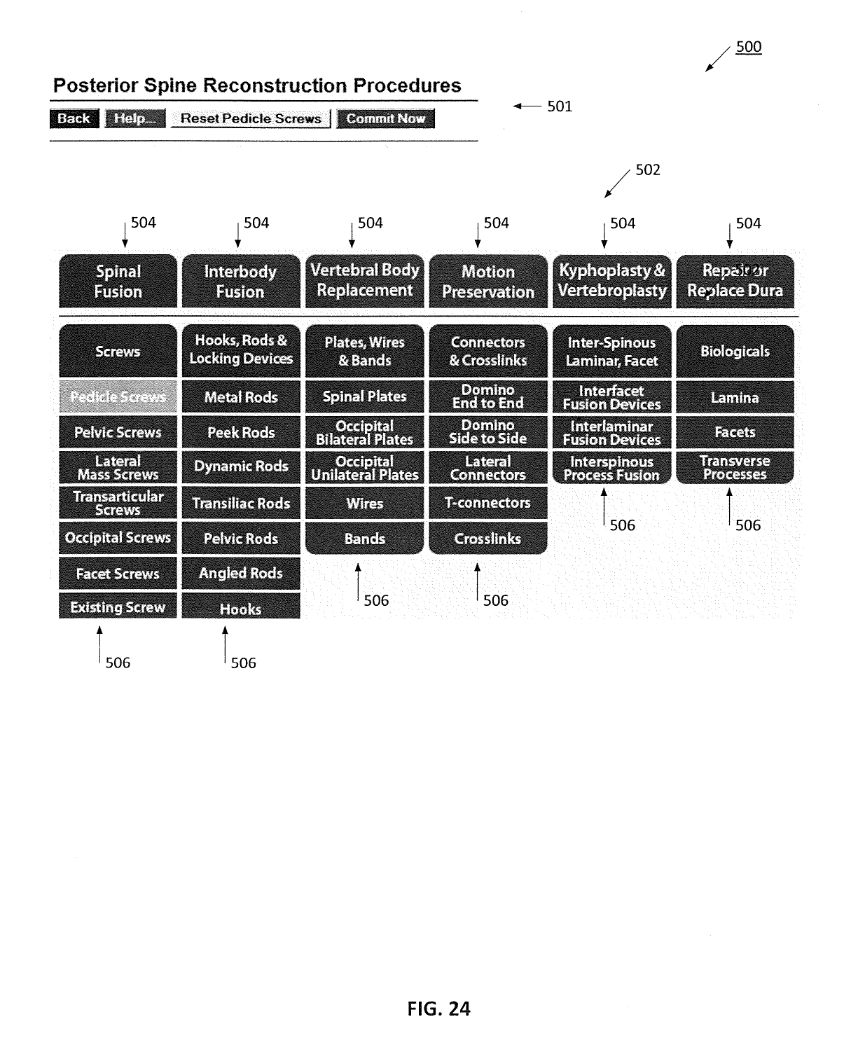

Referring now to FIG. 24, an input screen 500 is illustrated for a reconstruction method. The reconstruction method input 500 has a reconstruction selection area 502. It will be appreciated that many other types of selection arrangements may be used consistent with this disclosure. Generally, the reconstruction input 502 has a series of higher level selections 504, which then have columns 506 vertically aligned that contain more detailed information for the specific type of reconstruction selected. It will be understood that when a particular tool is selected, such as screws, further dialog boxes may be used for particular selection of the precise type of screw used. A similar set of dialog boxes may be provided for other selections. As illustrated in selection area 502, the medical data system allows for many types of tools; for example, the placement of screws, rods, plates, hardware inter-connects, cross-links, spacers, biologicals, and other types of implant devices and materials. It will be appreciated that other types of tools may be provided. Generally, each specific type of medical tool is implemented using its own dedicated graphical layer. Associated with this layer are a set of rules or constraints which define how the operator is allowed to interact with that layer, and may provide for intralayer conformance as well as interlayer conformance. In one example, an additional layer could be used for indicating removal procedures. This would provide an alternative to the graphical removal procedure presented earlier. As illustrated at input area 501, the reconstructions referenced in section 502 are maintained as temporary files until the operator or surgeon confirm that the reconstruction has been accurately and completely captured. At that time, the operator activates a commit function that places the reconstruction data into permanent memory, as described earlier.

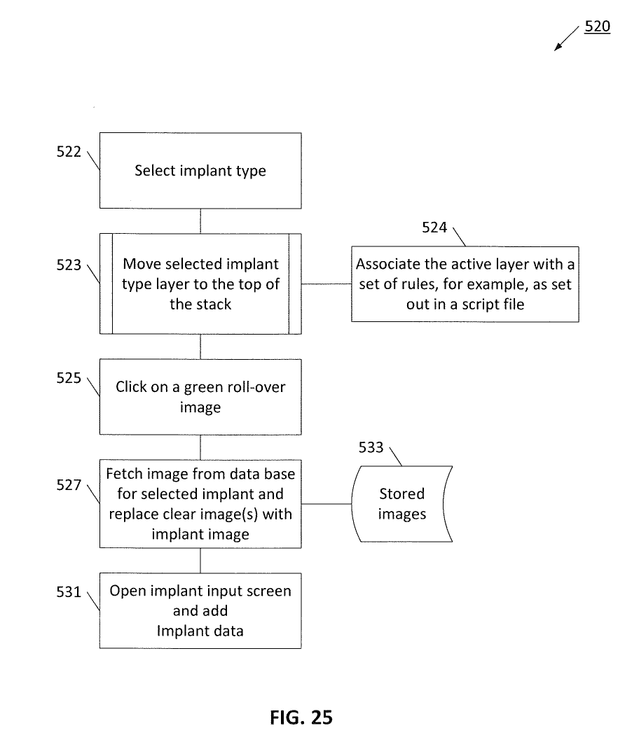

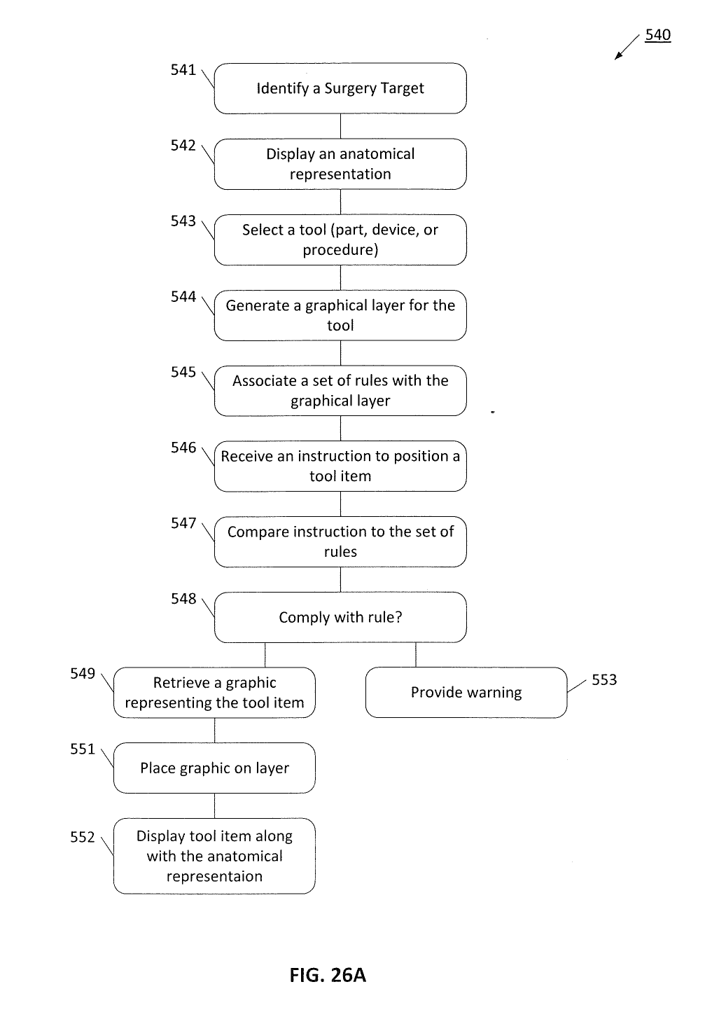

Referring now to FIG. 25, a reconstruction process 520 is illustrated. The operator selects a particular implant type as shown in block 522. The medical data system moves that graphical implant layer to the top of the graphic stack as shown in block 523. In many cases, the graphic layer intelligently assists the operator in proper placement of a tool. For example, the graphical layer may turn green when the operator rolls over an area that that particular tool may be placed, as shown in block 525. It will be understood that more intralayer and interlayer types of tests and constraints may be applied. As illustrated in block 524, each graphical layer applies a set of rules which may be, for example, implemented as a script file or other well-known software coding technique. Provided the operator has selected an authorized area to place a tool item, an image is retrieved from the stored images, as shown on blocks 527 and 533. The stored image representing the tool is then added to the layer, as shown on block 531, enabling the operator and the surgeon to confirm that the anatomical representation of the spinal surgery matches that which the surgeon has actually done.

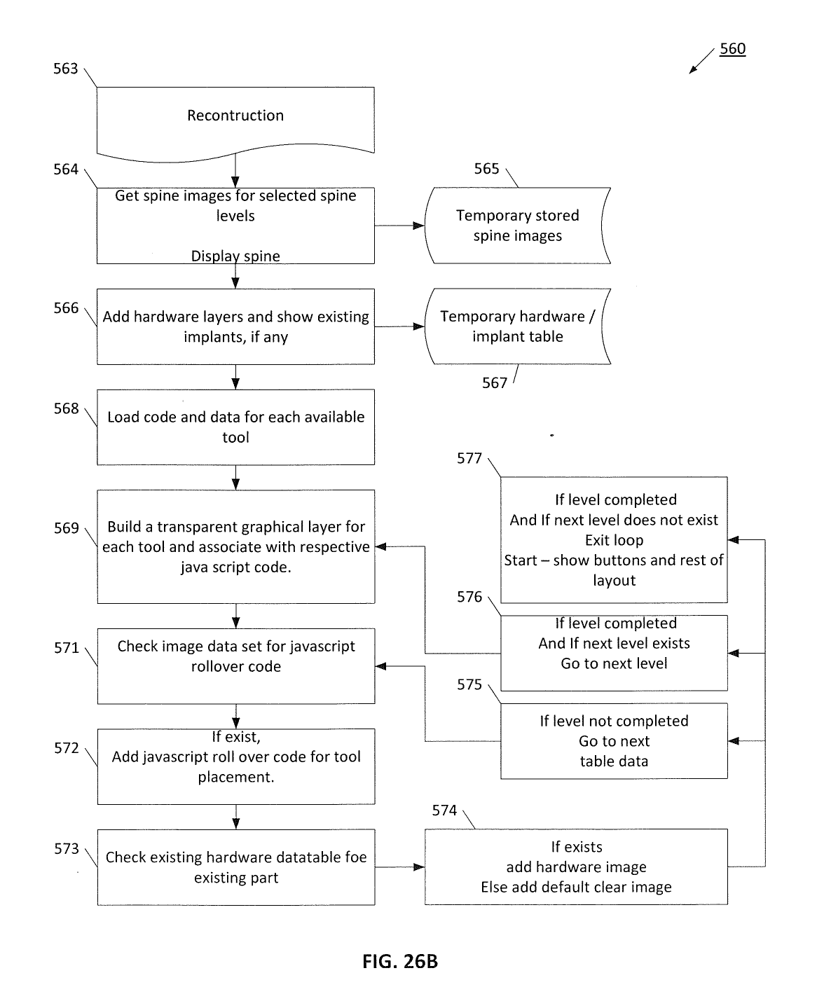

FIGS. 26A and 26B describe the general process of capturing implant information using the surgical data system. While performing the reconstructive operation, the doctor or surgeon determines a particular implant to use and a location for its positioning. Typically, a set of drop-down or menu selections will be used to indicate to the surgical data system what specific implant is going to be positioned and the specific type of implant that will be used. Upon selection of the implant that is going to be positioned, and appropriate annotation layer is activated and made accessible on the anatomical representation. This anatomical representation is intelligent and has restrictions as to the quantity and available locations for placing the particular implant. As, or immediately after the surgeon has positioned the implant, the computer operator may use the graphical input device to position that implant on the anatomical representation. The active intelligent layer restricts the placement of the implant to approved locations, and may provide a graphical feedback that a proper location has been located by the computer operator. For example, when a proper location has been found on the anatomical representation for the particular implant, that location on the anatomical representation may turn a different color, such as green. The layer may also intelligently confirm other indications that the implant may or may not be placed at that location. Once the computer operator is satisfied that the proper position has been found for the implant, the operator commits the location and the system places a graphical image of the implant at that location.

Advantageously, since the surgical data system has a display which may be seen by members of the surgical team, the doctors and the surgical team may immediately verify that the computer operator has properly place the implant. As previously illustrated, for example in FIG. 23, the anatomical representation of the spine shows various rods, screws, and connectors positioned on the spine, as well as indicating selected areas where tissue or bone has been removed. Using these intelligent layers, complete and accurate surgical data is collected, and is done in an intelligent and transparent way that reduces opportunities for mistake or fraud.

Referring now to FIG. 26A, a method 540 for graphically collecting data during the surgery is illustrated. Method 540 advantageously allows for the accurate, repeatable, and complete acquisition of near real time data during a surgery. It will be appreciated that method 540 may be supplemented with data received prior to the surgery, and supplemented with data after the surgery. Accordingly, method 540 focuses on the data collected during the surgery itself.

The first step of method 540 involves identifying a specific surgery target as indicated in box 541. The surgery target may be selected using textural input means, or may use a graphical input system. Both a tabular and graphical input of a spinal surgery selection was illustrated, for example, with reference to FIG. 10. The surgery target as described earlier may be, for example, a portion of a spine. It will be appreciated that many other types of surgeries are contemplated. In yet another example, the surgical process 540 can apply to other medical procedures other than surgery. Once the surgery target has been identified, the system displays an anatomical representation of the surgery target as illustrated in 542. Such an anatomical representation may be, for example, a section of spine 412 as illustrated in FIG. 18. It will be understood that other types of anatomical representations may be substituted. The operator then selects a particular tool to use with the surgical method as shown in block 543. The tool can represent a part, a device, or even a procedure. Typically, the part, device or procedure selection is selected to capture what the surgeon or other medical personnel are presently doing to the patient. For example, if the surgeon is inserting a screw into the spine, then the operator of the system 540 would select a screw layer so that they can select the proper screw and show its position on the surgery target. Medical devices may be for example, screws, rods, hardware, implants, or other item intended to be inserted into the human body. Other types of parts may include biomedical materials such as bone, skin, or epoxies and glues. Other tool layers may be used to document or illustrate medical procedures performed by the surgeon.

Typically, each tool will have its own associated graphical layer. It will be appreciated that these tools may be incrementally preloaded into a local memory system for faster access. By only sending incremental portions that have changed, or are new, bandwidth is preserved, thereby allowing the system to operate more efficiently and cost-effectively. In another example, the tools can be stored in other memory and retrieved as needed. In this way, the graphical layer for the tool may be generated in advance and made transparent, and only made active upon selection. In another example, the graphical layer is generated upon selection as shown in box 544. A set of rules is associated with the graphical layer as shown as 545. As discussed with reference to FIG. 19, the graphical layer may be divided into a matrix of action boxes, with each graphical cell useful for applying and assessing the rules. The operator of system 540 will instruct the system to place a tool item within the graphical layer as shown in 546. For example, if the surgeon places a screw into the patient's spine, the operator would select the screw tool layer, select the particular screw, and indicate its placement within the graphical layer. It will be understood that the order may be adjusted so the operator selects the location first, and then selects the particular type of screw. The system then compares the rules to the position selected by the operator as shown in 547. The system confirms that the operator's placement complies with the rule as shown in 548. Provided it does, a graphic representing that tool item is retrieved or activated as shown in 549, and then placed on the graphic layer as shown in 551. The graphic of the tool item is then displayed along with the anatomical representation as shown in 552. An example of an anatomical representation showing placements of screws, rods, and other hardware is shown and described with reference to FIG. 23. If however the operator has selected a position that does not comply with the rules, then a warning 553 is provided to the operator and surgeon.

Referring now to FIG. 26B, another method 560 is illustrated showing how a medical data system can capture surgical data regarding reconstruction as shown in block 563. As described earlier, the operator selects an area of the body, such as a section of spine, for the operation. That section of the spine is then retrieved from storage and displayed to the operator as shown in block 564. The level information is locally stored 565 until the operator has completed the procedure and committed the procedure to permanent storage. If this is a follow-up surgery, or a continuation of an ongoing surgery, there may be existing hardware identified for this patient as shown in 566. If so, those hardware layers are retrieved and displayed for the operator and the surgeon. Information regarding existing hardware is also temporarily stored as shown in 567. In method 560, all of the tool layers and their associated code that implements the rules are preloaded into a local memory as shown in 548. Doing so enables the system to react more efficiently to operator instructions. In doing so, the system preloads all of the graphical data layers into a stack, and then identifies all of the graphical layers as being transparent. In one example, the rules associated with each layer are implemented in Java Script as shown in block 569. When the operator selects a specific tool layer to operate on, it is moved to the top and given focus as shown in block 571. In this way, as the operator moves a mouse or other indicator around the graphical layer, the system can respond with graphical, textual, or audible indicators whether or not the current position of the curser is authorized according to the java script rules as shown in block 572.

Once the operator selects replacement, the system confirms that the position is authorized, and identifies the part as being authorized as shown in 573 and extracts an image indicative of the part as shown in box 574. If the operator desires to do more work using this graphical layer, then that layer remains focused and additional hardware may be added as indicated in box 575. In another example, the operator may move to operate using a different tool as shown in 576. In this case, a new layer is moved to the top and the prior layer is moved to its pre-defined vertical relationship within the vertical overlay stack. Finally, if the user has completed the reconstruction aspect of the surgery as shown in block 577, then the reconstruction process is completed and the operator can continue on to other aspects of the process, such as generating the automated surgery report.

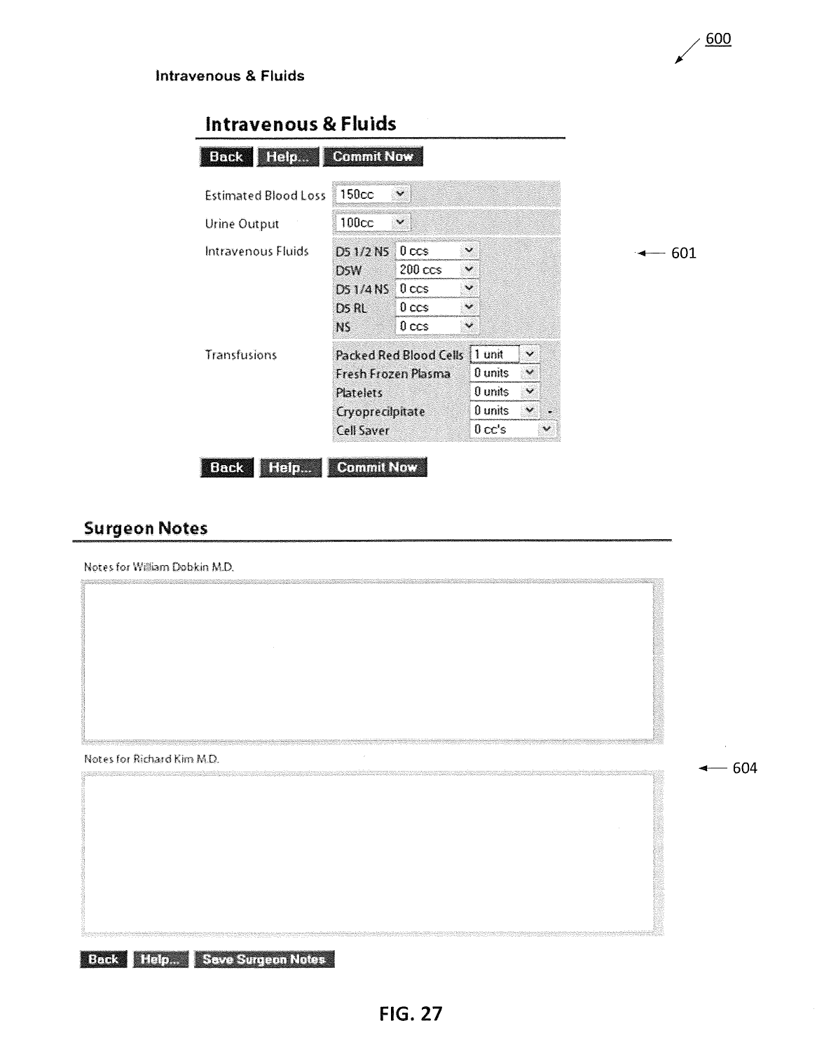

Referring now to FIG. 27, example input screens 600 for the surgical data system are illustrated. The surgical data system also allows for capture or use of the specific intravenous and fluid substances 601 used for the medical procedure. This includes both pharmaceuticals and blood. Additionally, the surgeon is allowed to enter free-form notes 604 in the system, which may be done textually or through dictation.

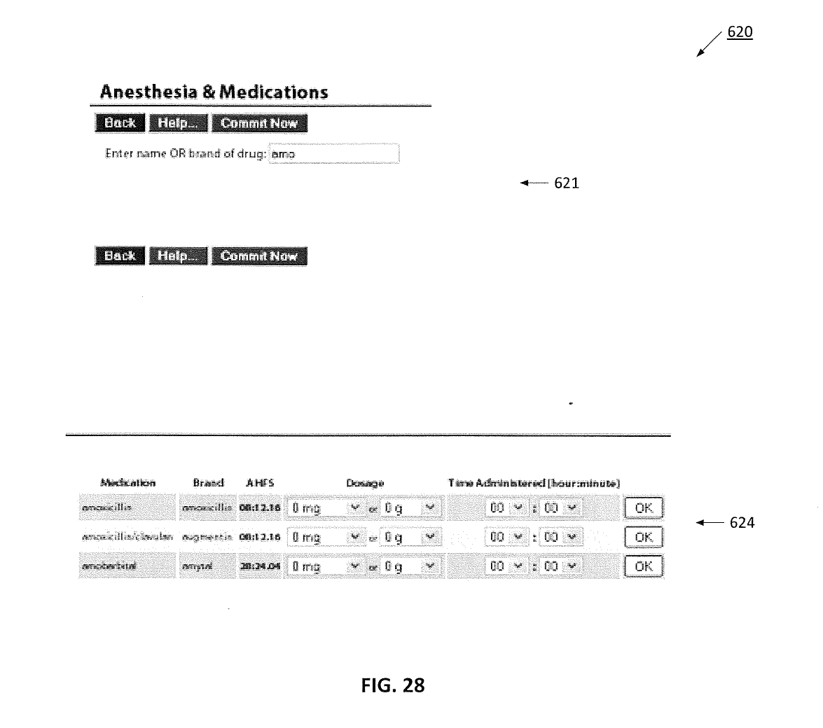

Referring now to FIG. 28, example input screens 620 for the surgical data system are illustrated. The surgical data system allows for capture of anesthesia and medications 621 used during the surgery, as well as to track specific times and dosages 624 used with that particular patient.

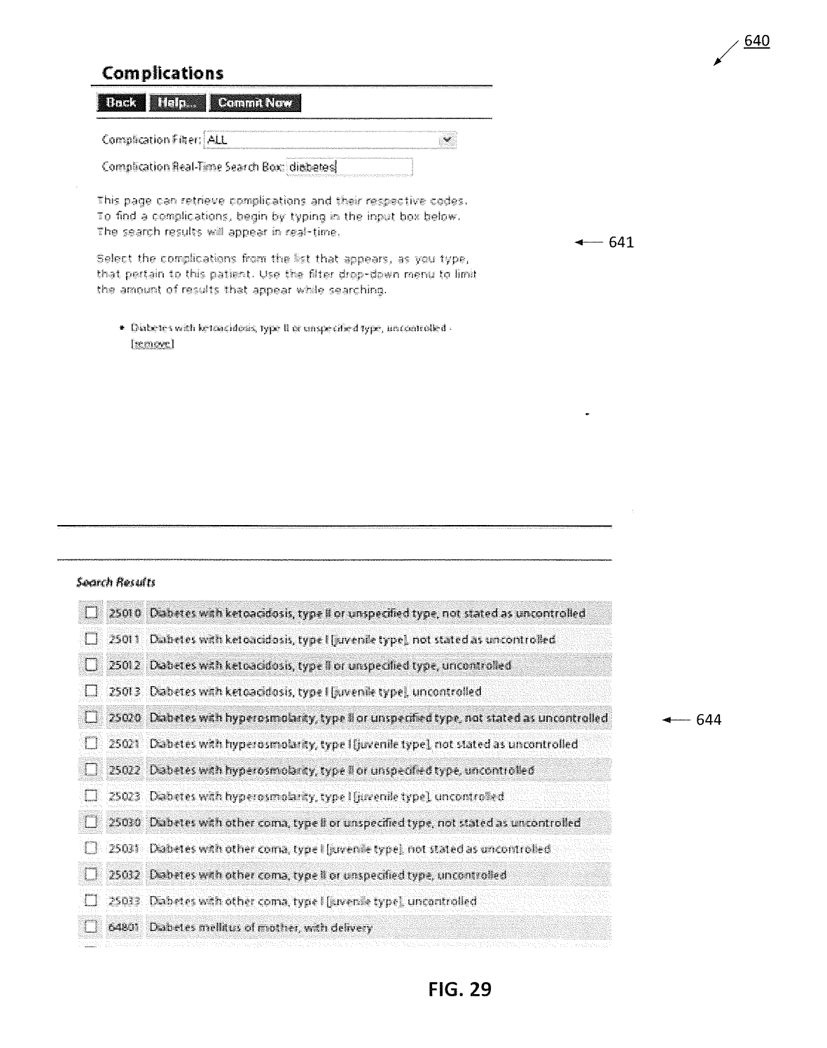

As illustrated in example input screens 640 of FIG. 29, the surgical data system includes a drop-down list 644 of common complications that occurred during the particular type of surgery type selected, which the computer operator may enter in real time as they occur. Also, the surgical data system contemplates that particular unique complications may be identified and entered during the surgery. It is also contemplated that complications may be added or supplemented by the surgeon during the process of approving the operative report. Due to the large number any types of complications, a selection input screen 641 is provided to limit the number of specific choices presented in the selection area 644. It will be appreciated that the available complications may comply with hospital or billing standards to facilitate efficient billing and invoicing.

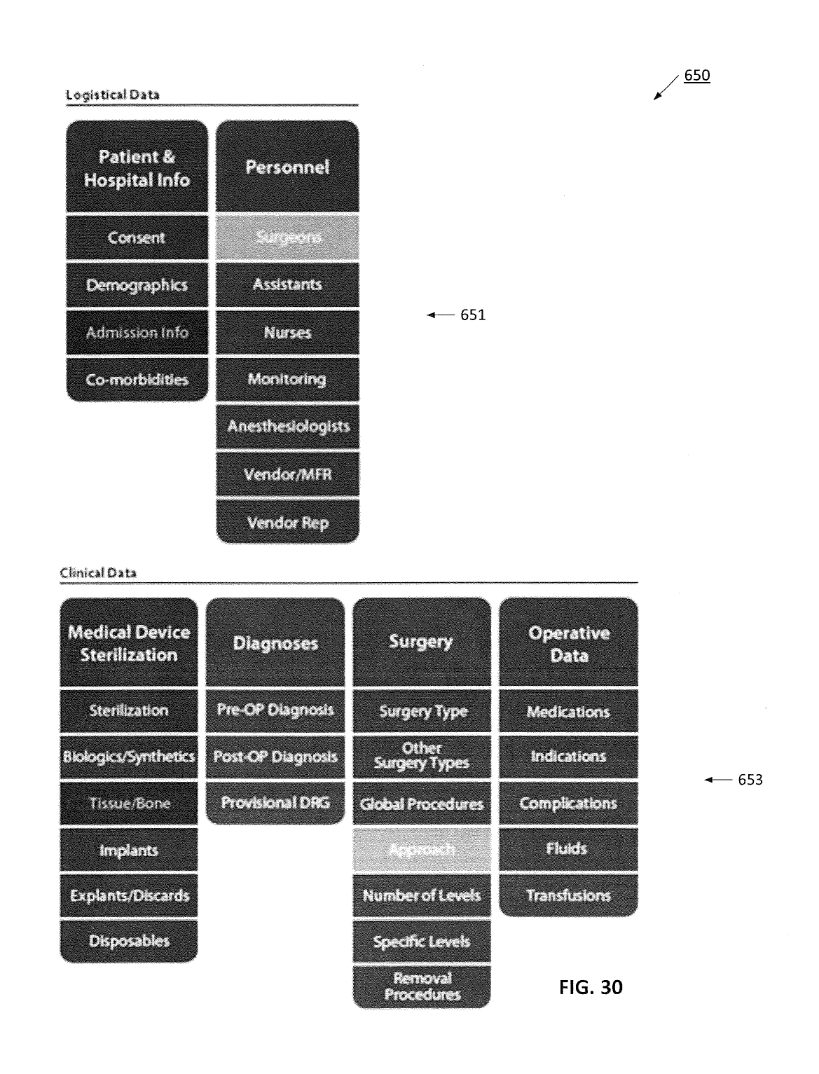

Referring now to FIG. 30, example input screens 650 for the surgical data system are illustrated. Other information may be added into the medical data system for more complete collection of data regarding a medical procedure. It will be understood that some of this data may be collected prior to the medical procedure; for example, by the doctor or nurse during patient intake. It will also be understood that certain of this information may be added by the doctor prior to the surgery, and that some of it may be added or amended within the surgical time. Further, it will be appreciated that more or less information may be collected depending on application-specific needs. Input screen 650 may be divided into logical sections as illustrated at 651 and 653. It will be understood that a wide range of arrangements may be made for allowing data input that are consistent with the medical data system. Advantageously, and consistent with the general input processes already described for the medical data system, the input screens 650 are logically arranged to guide and assist the user in the complete and accurate capture of procedure information.

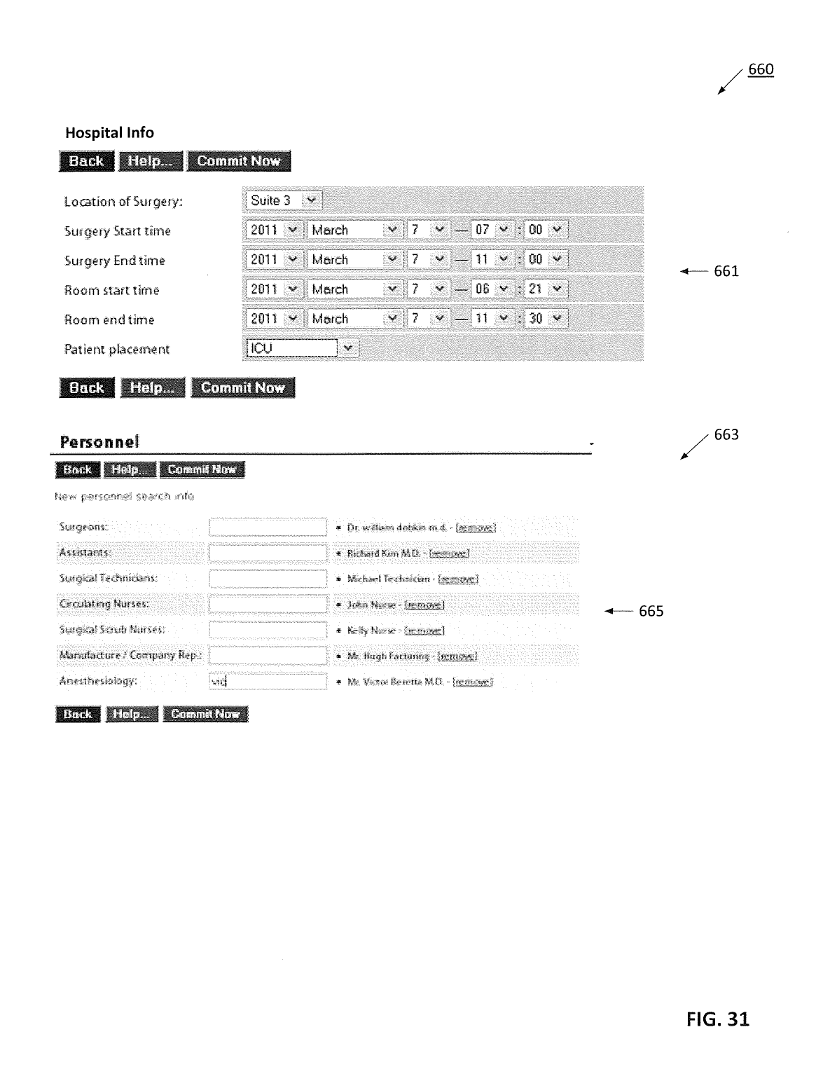

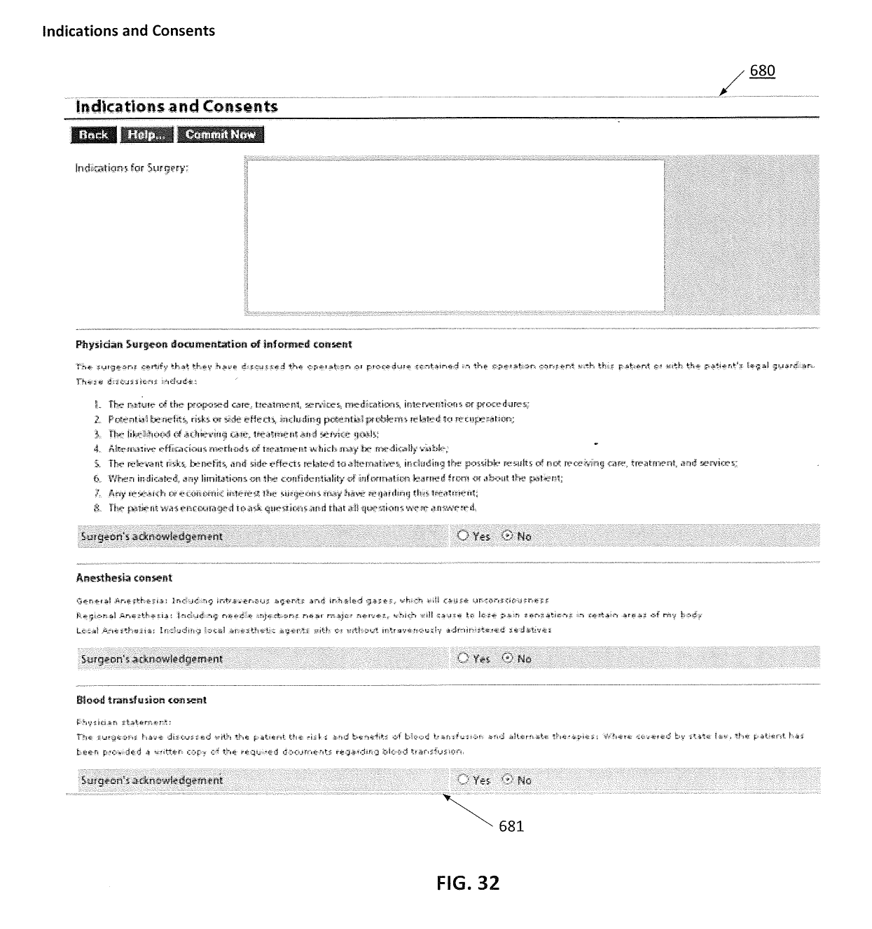

FIG. 31 shows example input screens 660 for the surgical data system. For example, the location 661 of the surgery may be collected, including information regarding the time and duration for the operation. Further, the system may collect personnel information 663 in a set of information blocks 665. In this way, all or at least the significant personnel involved with delivering the medical procedure may be permanently tracked. As illustrated in FIG. 32, additional indications and consents 680 may be collected from the patient. Such a document is useful for tracking the legal authorization to perform the surgery. The indications input screen also collects data as to whether the surgeon has acknowledged the consents, as shown at area 681.

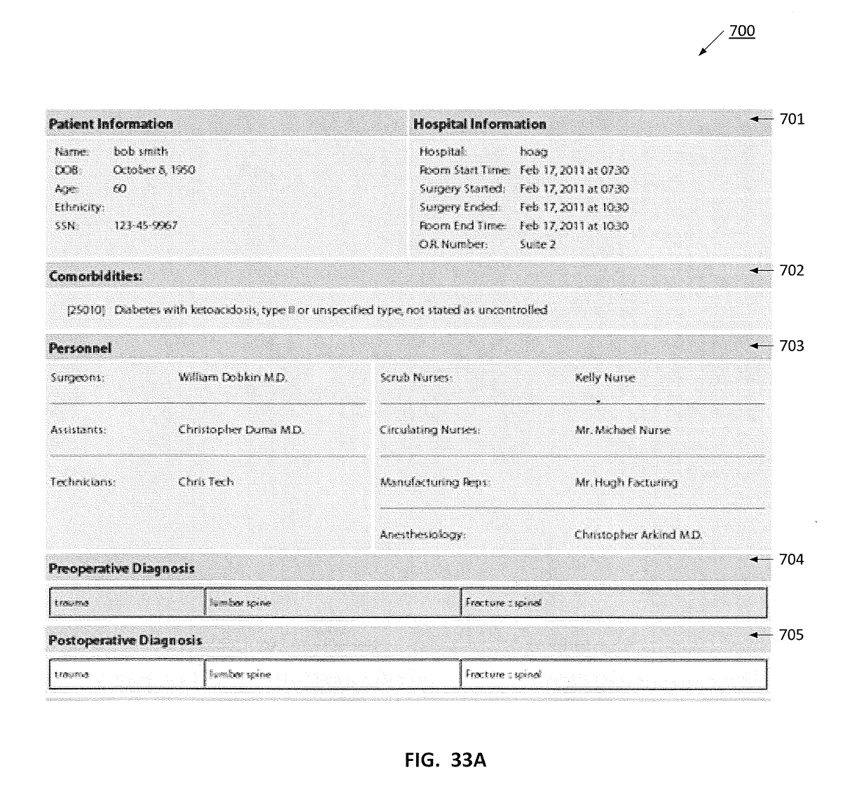

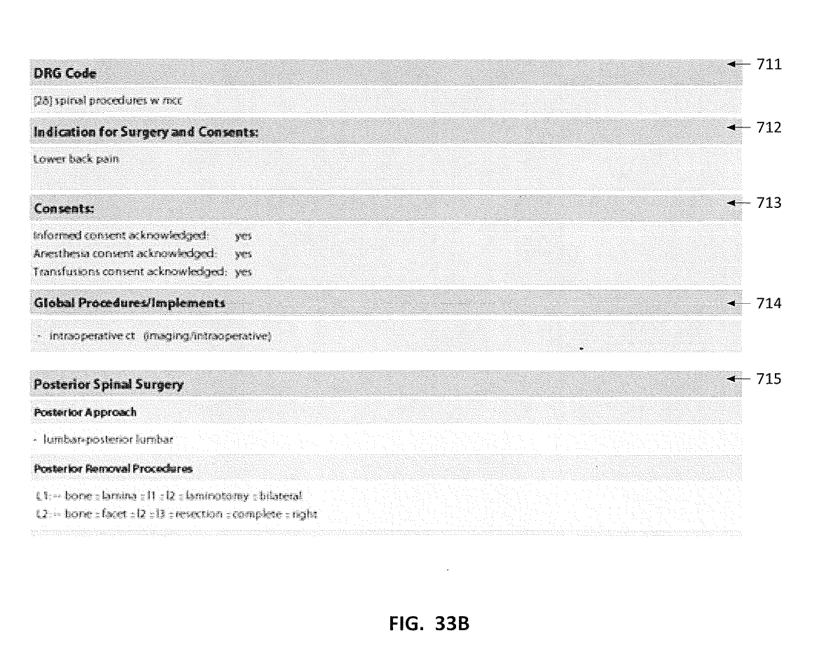

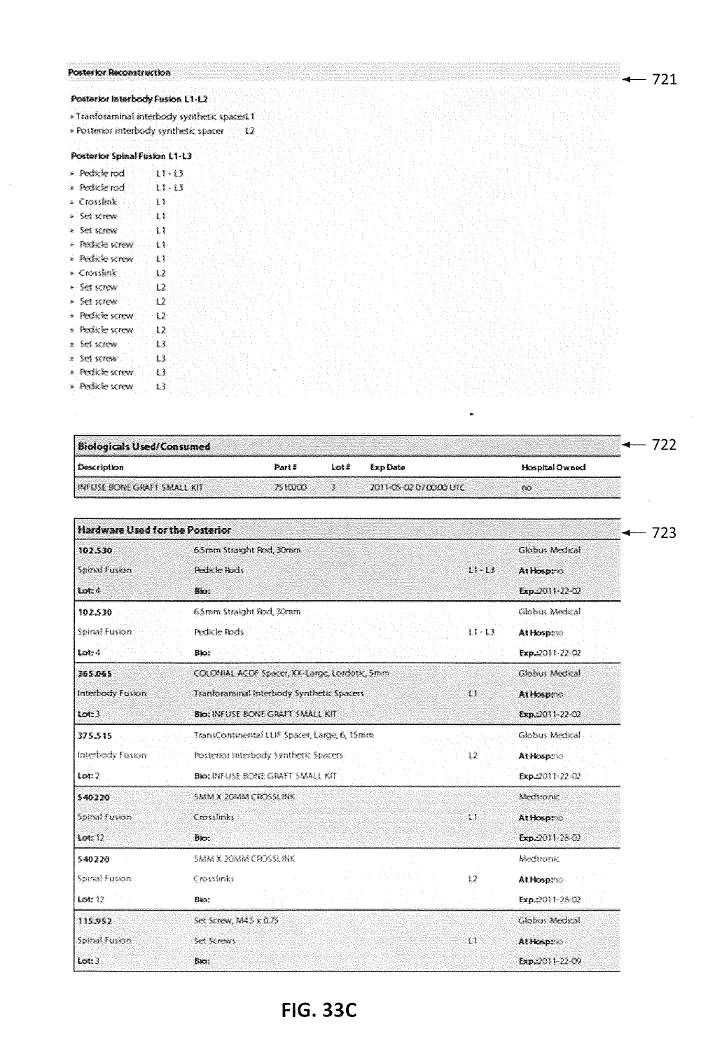

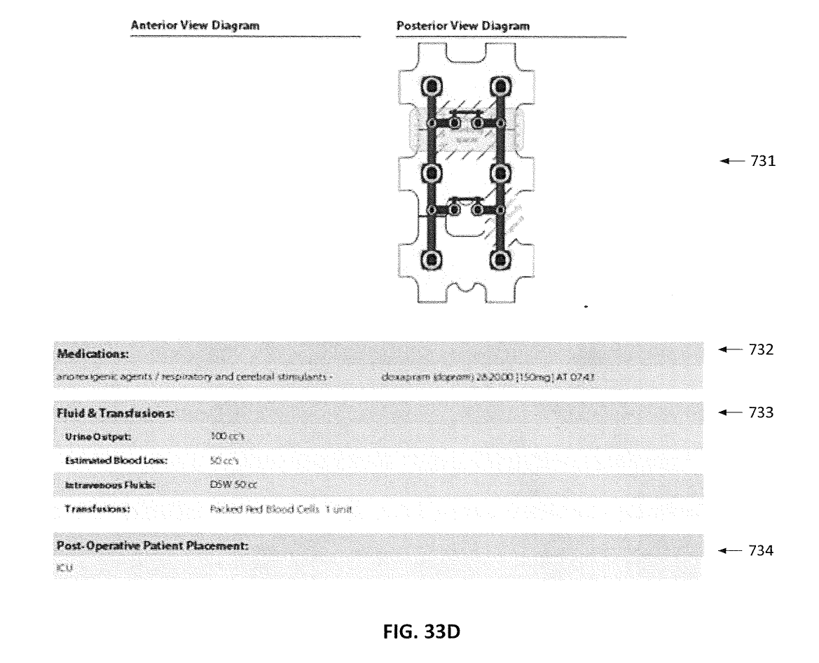

FIGS. 33A-D illustrate how the medical data system can be used to generate an automated operative report. In particular, these pages illustrate a preview of the operative report. This document is generated after the surgeon has completed input into the operative report generator, but prior to committing the surgical data to the permanent patient record. These documents provide a complete review of the medical procedures and surgical processes performed, and include the annotated anatomical representation which was built during the operation. Once the surgeon or medical manager is satisfied that the operative report is correct, the doctor or medical manager commits the surgical data and the final operative report is generated. Concurrently, the temporary surgical file is closed and the surgical data committed to the patient and hospitals' permanent records. Advantageously, the generation of the operative report is highly automated and efficient for the doctor to generate. As will be understood, the specific form and content of the operative report may be adjusted according to application needs, the particular medical procedure, or the particular surgery performed.

Referring still to FIGS. 33A through 33D, the automatically generated operative report is illustrated. It will be appreciated that the operative report may contain more or fewer amounts of information, depending upon application-specific needs. During the surgery, information is collected by the medical data system into a temporary local storage. Upon completion of the surgery, the surgeon or medical procedure manager will confirm that the medical data system has accurately collected all information regarding the patient, the procedure environment, and the outcomes of the surgery. Upon authorization, the surgeon or operator of the system will commit the entire operation and patient information to a permanent operative report. Once committed, it is intended that the operative report become permanent and unalterable. Alternatively, the operative report may be altered, but any indications of alteration of changes would be noted within the document itself. Immediately upon committing the medical procedure information to the permanent database, a final operative report becomes available for distribution and use. The operative report is the primary document used by the doctor and the medical facility to document the specific procedures, and is used to drive the billing, inventory, invoicing, and business needs of the medical facility. The operative report 700 may include hospital information 701, comorbidity information 702, specific personnel relevant to providing the medical procedure 703, the physician's pre-operative diagnosis 704, and the post-operative diagnosis 705. As illustrated in 33B, the operative report may provide the specific DRG code for billing and inventory purposes, along with indications 712 and consents 713. The operative report also identifies data such as global procedures 714, and then gives specifics about the surgery performed 715. Referring to 33C, a tabular description of the reconstruction 721 is identified, along with specific, trackable information regarding biologicals used 722. Similar detailed information is provided for every screw, rod, connector, or other implant used 723. The operative report also maintains a version of the anatomical representation 731 as shown on FIG. 33D. The complexity of the multi-layer systems used during data collection may be collapsed into a simple single flat picture for convenience and for resistance to tampering. The operative report may also identify any medications used 732, any blood or transfusions 733, and indication of where the patient was moved to immediately after surgery 734. It will be appreciated that other information may be collected and presented consistent with this disclosure.

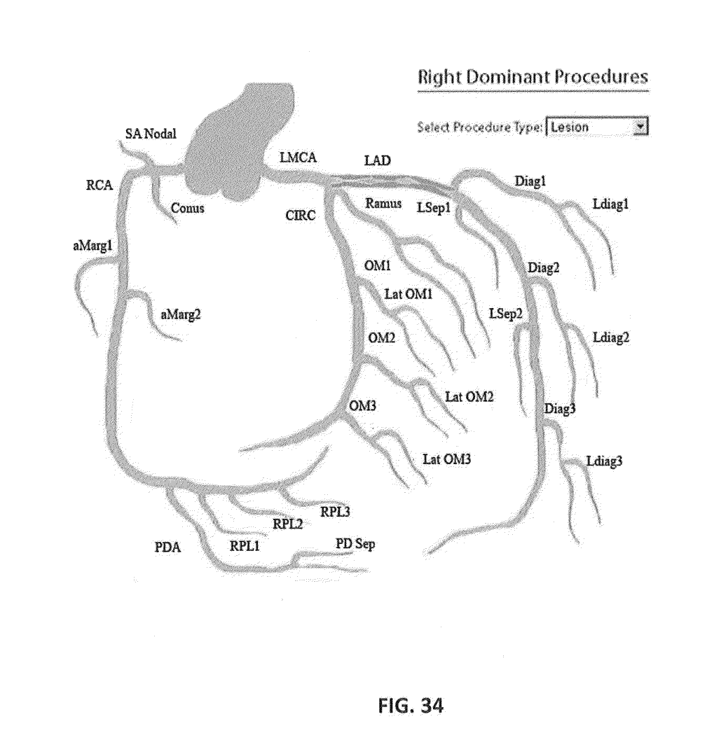

As illustrated in FIG. 34, the medical data system is useful for other types of medical procedures or surgeries. Upon selection of a cardiac surgery type, an anatomical representation of a heart or pulmonary system may be illustrated. As with the spinal surgery type, selection of a specific aspect from a graphical or tabular representation of the heart system may be retrieved or built and an anatomical representation of that specific portion can be displayed that represents the target of the surgery. For example, this FIG. 34 shows that a particular aspect of the heart system has been selected for a medical procedure. In accordance with this invention, an anatomical representation of the surgical site is presented, and an operator selects and places a stint to represent the actual surgical procedure performed. In this way, the anatomical representation of the heart is annotated in near real time to reflect occurrences in the operating room. Here, the anatomical representation of the heart has been annotated to reflect the type size, and location for a cardiac stint.

The surgical data system supports a wide range of analytics for the collected data. It will be appreciated that any number of formats and data presentations may be used by the surgical data system. It will also be appreciated that the data collected by the surgical data system may be exported to other analytical tools for further analysis, use, or distribution. The surgical data system maintains detailed records of the specific disposables, parts, tools, implants, and biologicals used for a particular surgery or patient. Accordingly, once the operative report is complete, the system may automatically generate communications to hospital staff or vendors to replenish inventories. Advantageously, the process of maintaining sufficient stock on disposables, as well as facilitating timely issuing of invoices and payment of bills is accomplished using the surgical data system. The surgical data system also contemplates highly automated communications with vendors, thereby facilitating restocking and vendor billing area. Advantageously, the vendor also is provided a simplified process for assuring stock for a particular part is maintained. The medical data system enables confidential sharing of implant, surgical, and patient data among the various parties that provided medical care and supply equipment.

While particular preferred and alternative embodiments of the present invention have been disclosed, it will be appreciated that many various modifications and extensions of the above described technology may be implemented using the teaching of this invention. All such modifications and extensions are intended to be included within the true spirit and scope of the appended claims.

What is claimed, is:

1. A method for graphically collecting surgical data indicative of a surgery being performed in a surgery room, comprising:
identifying a surgery target representing a portion of a human body;
displaying, in the surgery room, an anatomical representation of the surgery target using a base graphical layer;
selecting a tool from a set of available tools, the available tools each reflecting a medical part, medical device, or medical procedure;
providing a graphical layer representing the selected tool, the graphical layer having an associated rule;
receiving an instruction from a user to position a tool item on the graphical layer;
confirming that the position in the received instruction satisfies the rules;
retrieving a graphical image indicative of the tool item;
placing the graphical image on the graphical layer according to the position in the instruction; and
displaying, in the surgery room, the graphical image along with the base graphical layer.

2. The method according to claim 1, further including identifying the surgery type as a spinal, heart, hip, knee, shoulder, brain, eye, thoracic, or cosmetic surgery.

3. The method according to claim 1, further including selecting a tool for placing an implant into a patient.

4. The method according to claim 1, further including generating a graphical layer for a plurality of the available tools and preloading the generated layers.

5. The method according to claim 1, further including generating the graphical layer responsive to selecting the tool.

6. The method according to claim 1, further including:
associating a plurality of the available tools with respective rules; and
preloading the rules.

7. The method according to claim 1, further including:
retrieving a set of tool items responsive to selecting the tool; and
selecting the tool item from the set of tool items.

8. The method according to claim 7, further including displaying, in the surgery room, information indicative of the expiration date, lot number, tray number or sterilization history for the tool item.

9. A method for graphically collecting surgical data indicative of a spinal surgery being performed in a surgery room, comprising:
displaying, in the surgery room, an anatomical representation of a portion of the spine that is to be operated on;
selecting a tool from a set of available tools, the available tools reflecting a screw, a rod, an adhesive, a biomaterial, or an adhesive procedure;
providing a graphical layer representing the selected tool, the graphical layer having an associated rule;
receiving an instruction from a user to position a tool item on the graphical layer;
confirming that the position in the received instruction satisfies the rule;
retrieving a graphical image indicative of the tool item;
placing the graphical image on the graphical layer according to the position in the instruction; and
displaying, in the surgery room, the graphical image along with the base graphical layer.

10. The method according to claim 9, further including generating a graphical layer for a plurality of the available tools and preloading the generated layers.

11. The method according to claim 9, further including generating the graphical layer responsive to selecting the tool.

12. The method according to claim 9, further including:
associating a plurality of the available tools with respective rules; and
preloading the rules.

13. The method according to claim 1, further including:
retrieving a set of tool items responsive to selecting the tool; and
selecting the tool item from the set of tool items.

14. The method according to claim 13, further including displaying, in the surgery room, information indicative of the expiration date, lot number, tray number or sterilization history for the tool item.

15. The method according to claim 9, further including dividing the graphical layer into a matrix of action boxes, the rule using the action boxes to assess compliance.

16. The method according to claim 15, further including receiving the instruction to position the tool item in one of the action boxes, and displaying the graphical image in that action box.

17. The method according to claim 9, further including generating and presenting a warning if the position in the received instruction does not satisfy the rule.

18. The method according to claim 9, further including:
storing the graphical image in a temporary storage;
receiving and instruction from the user that the graphical image is properly displayed; and
storing the graphical image into permanent storage.
